(19) United States
(12) Patent Application Publication
(10) Pub. No.: US 2007/0122482 A1
(43) Pub. Date: May 31, 2007

(54) Method for preparing modified release pharmaceutical compositions

(76) Inventors: Per Holm, Vanlose (DK); Tomas Norling, Lyngby (DK)

Correspondence Address: CHERYL H AGRIS PHD, PO BOX 806, PELHAM, NY, 10803, US

(21) Appl. No.: 10/574,125

(22) PCT Filed: Oct. 4, 2004
(86) PCT No.: PCT/DK04/00669

§ 371 Date: Jun. 13, 2006

(30) Foreign Application Priority Data
Oct. 3, 2003 (DK) PA 2003 01459

(51) Int. Cl. A61K 9/28; A61K 9/14

(52) U.S. Cl. 424/489; 427/2.14

(57) ABSTRACT

A method for the preparation of a pharmaceutical particulate composition for modified release of one or more therapeutically, prophylactically and/or diagnostically active substances, the method involving spraying of a composition comprising an oily material on a solid composition in order to subject the solid composition to a controlled agglomeration process, whereby individual particles are aggregated into agglomerates in a controlled manner and a relatively small particle size and particle size distribution is obtained, the particulate composition comprising a sufficient amount of at least one release-rate modifying substance to provide a modified release of the active substance sufficient to provide duration of therapeutic, prophylactic and/or diagnostic effect of at least about 2 hours when the composition is exposed to an aqueous environment.

### FIELD OF THE INVENTION

The present invention relates to a method for the preparation of a pharmaceutical particulate composition for modified release of one or more therapeutically, prophylactically and/or diagnostically active substances, the method involves spraying of a composition comprising an oily material on a solid composition in order to subject the solid composition to a controlled agglomeration process, i.e. a process whereby individual particles are aggregated into agglomerates in a controlled manner in order to obtain a desired and relatively small particle size and particle size distribution. The particulate composition obtained may be used as such or it may be further processed into a solid dosage form e.g. in the form of a so-called mono- or polydepot composition from which the release of the active substance takes place.

The particulate composition or the solid dosage form according to the invention comprises a sufficient amount of at least one release-rate modifier to provide a modified release of the active substance sufficient to provide duration of therapeutic, prophylactic and/or diagnostic effect of at least about 2 hours when the composition is exposed to an aqueous environment.

The invention also relates to modified release pharmaceutical compositions comprising the particulate composition according to the invention.

### BACKGROUND OF THE INVENTION

The development of modified-release compositions have a clinical rational as it may reduce dose related side effects, improve efficacy and add to compliance to drug therapy. Modified release products may be developed to reduce dose frequency, which adds to convenience of use, which in turn may facilitate compliance. Another rationale for developing modified release preparations is smoothing the peaks of the plasma concentration curves (slow release) in order to prevent peak concentration related adverse events.

The combination of a bioavailability enhancing formulation of a poorly soluble drug with modified release is normally considered to be obsolete. The combination is contradictive since modified release normally result in lower bioavailability. However, if the bioavailability is only slightly decreased, the clinical benefits mentioned above might add to the general advantage of bioavailability enhancing formulations. The present inventors have invented a method for the preparation of particulate material into which relatively large amounts of an oily material can be incorporated by means of a so-called novel controlled agglomeration technique. This invention is described in WO 03/004001, which is hereby incorporated by reference. The controlled agglomeration process has been found to be especially suitable for use for active substances having a poor bioavailability and/or poor water-solubility in that the bioavailability can be markedly increased and the dissolution of the active substance from the particulate material can be significantly enhanced. The present invention is focused on the use of the controlled agglomeration technique in order to overcome the generally recognized problems in the development of modified release pharmaceutical compositions. For example due to the biopharmaceutical properties of a drug substance drug (e.g. poor water-solubility and/or bioavailability) it may be impossible to obtain the desired effect by administering the drug substance in the form of a controlled release composition even if the drug substance is a candidate for administration as a controlled release composition, i.e. the drug substance has e.g. a relatively short half-life. it may be subject to first pass metabolism or it may be desired to prolong the therapeutic effect after a sigle administration in a long-term treatment (e.g. chronic disease) situation. However, subjecting an active substance to the controlled agglomeration technique increases the dissolution and/or the bioavailability to such an extent that it now is possible and realistic to provide modified release compositions comprising active substances that normally.are not considered as suitable for formulation into modified release compositions. The present invention is not limited to such active substances but is a general technique that can be used to all kinds of active substances irrespective their water-solubility and/or bioavailability.

### DISCLOSURE OF THE INVENTION

As used herein, the term “drug” means a compound intended for use in diagnosis, cure, mitigation, treatment, or prevention of disease in man or other animals.

In this context, the term “dosage form” means the form in which the drug is delivered to the patient. This could be parenteral, topical, tablet, oral (liquid or dissolved powder), suppository, inhalation, transdermal, etc.

In the present context, the terms “controlled release” and “modified release” are intended to be equivalent terms covering any type of drug release from a composition prepared according to the invention, which drug release is appropriate to obtain a specific therapeutic or prophylactic response after administration to a subject. A person skilled in the art knows how controlled release/modified release differs from the release of plain tablets or capsules. The terms “release in a controlled manner” or “release in a modified manner” have the same meaning as stated above. The terms include slow release (that results in a lower Cₘₐₓ and later tₘₐₓ, but t_{1/2}is unchanged), extended release (that results in a lower Cₘₐₓ, later tₘₐₓ, but apparent t_{1/2} is longer); delayed release (that result in an unchanged Cₘₐₓ, but lag time and, accordingly, tₘₐₓ is delayed, and t_{1/2} is unchanged) as well as pulsatile release, burst release, sustained release, prolonged release, chrono-optimized release, fast release (to obtain an enhanced onset of action) etc. Included in the terms is also e.g. utilization of specific conditions within the body e.g. different enzymes or pH changes in order to control the release of the drug substance.

In this context, the term “erosion” or “eroding” means a gradual breakdown of the surface of a material or structure, for example of a tablet or the coating of a tablet.

In the present context, the terms “modified release” or “controlled release” indicate that efforts have been made to deliberately or actively target a specified release of an active drug substance from a drug composition. Basically any type of altered release pattern is included in these terms.

The terms could theoretically be applied to any therapeutic administration of a drug, but in this context it is limited to oral administration of solid dosage forms. The terms modified or controlled release cover a number of sub-terms such as, e.g., slow-release, extended-release, prolonged-release, sustained-release, delayed-release, pulsed-release and also site-specific releases like: buccal-release, gastrointestinal-release, stomach-release, intestinal-release, duodenal-release, jejunum-release, ileum-release and colon-release.

For site-specific releases, including delayed release and pulsed release, a fast release at a specific site or at predetermined time would normally be attractive. The delaying factor could be a coating or a sensitive matrix withholding releaseAuntil a certain time had passed, pH had changed, the composition had been subject to enzymes, or some other site-specific external factor is present. Ion-exchange systems would also be considered members of this class.

In the group of slow-release, the controlling factors most often are either a semi-permeable coating or a gelling barrier of some kind. The systems can be either mono-particulate (matrix) or multi-particulate (granules, pellets, beads, etc.).

With respect to the compositions according to the present invention, the modified release compositions of this invention can be widely implemented and all the types of formulation principles mentioned above and in the following can be employed provided that the basic material is a particulate composition that has been subject to a controlled agglomeration process as described in WO 03/004001 (by the present inventors). Accordingly, the advantages obtained by the method according to the present invention are based on the advantages obtainable by a controlled agglomeration process in combination with the advantages obtainable by employing controlled release techniques.

In one aspect, the invention relates to a method for the preparation of a pharmaceutical particulate composition for modified release of one or more therapeutically, prophylactically and/or diagnostically active substances, the method comprising spraying a first composition comprising an oily material, which has a melting point of about 5° C. or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C., at least about 5° C. or at least about 10° C. lower than the melting point of the oily material and/or of the first composition, optionally, mixing or other means of mechanical working the second composition onto which the first composition is sprayed to obtain a particulate material, optionally, adding one or more release-rate modifier, mixing or other means of mechanical working the second composition—including, if relevant, the added one or more release-rate modifying substances—onto which the first composition is sprayed to obtain a particulate composition, the particulate composition comprising a sufficient amount of at least one release-rate modifier to provide a modified release of the active substance sufficient to provide a duration of therapeutic, prophylactic and/or diagnostic effect of at least about 2 hours such as, e.g., at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 15 hours, at least about 17 hours, at least about 20 hours, at least about 22 hours or at least about 24 hours when the composition is exposed to an aqueous environment.

In another aspect, the invention relates to a method for the preparation of a pharmaceutical particulate composition for modified release of one or more therapeutically, prophylactically and/or diagnostically active substances, the method comprising spraying a first composition comprising an oily material, which has a melting point of about 5C or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C., at least about 5° C. or at least about 10° C. lower than the melting point of the oily material and/or of the first composition, optionally, mixing or other means of mechanical working the second composition onto which the first composition is sprayed to obtain a particulate material, optionally, adding one or more release-rate modifier, mixing or other means of mechanical working the second composition—including, if relevant, the added one or more release-rate modifying substances—onto which the first composition is sprayed to obtain a particulate composition, the particulate composition comprising a sufficient amount of at least one release-rate modifier to provide a modified release of the active substance sufficient to provide a dissolution rate in vitro of the particulate composition, which—when measured according to USP dissolution test (paddle) employing water as dissolution medium, 100 rpm and a temperature of about 37° C. permits release of less than 85% w/w within about 30 minutes after start of the test.

In a further aspect, the invention relates to a method for the preparation of a pharmaceutical particulate composition for modified release of one or more therapeutically, prophylactically and/or diagnostically active substances, the method comprising spraying a first composition comprising an oily material, which has a melting point of about 5° C. or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C, at least about 5° C. or at least about 10° C. lower than the melting point of the oily material and/or of the first composition, optionally, mixing or other means of mechanical working the second composition onto which the first composition is sprayed to obtain a particulate material, optionally, adding one or more release-rate modifier, mixing or other means of mechanical working the second composition—including, if relevant, the added one or more release-rate modifying substances—onto which the first composition is sprayed to obtain a particulate composition, the particulate composition comprising a sufficient amount of at least one release-rate modifier so that following ingestion by a subject in need thereof the active substance is released in the gastrointestinal tract of the mammal at a rate so that less than 85% w/w is released within the first 30 minutes after ingestion.

As it appears from the above, the three different aspects involve the same general process steps. However, the requirements fulfilled by the particulate composition differ. Within the scope of the invention is also any combination of these three aspects, e.g. where the particulate composition fulfils one, two or all three requirements.

In a method according to the invention an important issue is incorporation of one or more release-modifying substances. From the description above, it is clear that such a substance may be incorporated at any time during the process. Thus, the release-rate modifying substance may be present in the first and/or second composition and then being an integrated part of the controlled agglomeration process or it may be added to the particulate material resulting from the controlled agglomeration process (i.e. from the controlled building up of particle size of the second composition and incorporation of a relatively large amount of an oily material into the second composition). In these situations, the modified release composition may be presented in the form of a modified release particulate material, i.e. a matrix multi-particulate material comprising a plurality of drug-containing particles, each particle comprising a mixture of the active substance with one or more excipients selected to form a matrix capable of releasing the active substance in a predetermined manner. The matrix multi-particulate material may be coated and it may be processed into suitable dosage forms dependant on the desired administration route. Thus, suitable dosage forms for oral administration encompasses the so-called multiple-unit dosage forms including powders, sachets, capsules and tablets (which may also be coated). The modified release compositions may also be presented in the form of a single-unit dosage form, wherein the active substance is embedded or dispersed in a matrix that serves to control the release of the active substance into an aqueous environment. When the active substance is embedded or dispersed in a matrix, the release of the active substance predominantly takes place from the surface of the matrix. In the single-unit dosage form, the particulate material is typically presented in the form of tablets or capsules. Such dosage forms may be provided with a coating.

Other kinds of modified release compositions may also be provided according to the invention. Thus, as it is clear from the above discussion, the one or more release-rate modifying substance may also be applied in the form of a coating. Thus, the individual particles obtained after a controlled agglomeration process may be coating with a coating comprising one or more release-rate modifying substance or the particulate material may be processed into a suitable dosage from that subsequently is coated with a a coating comprising one or more release-rate modifying substance. In those cases where a semi-permeable membrane is applied as a coating material, e.g. by use of a water-insoluble but water-diffusable film-forming material, the release of the active substance from the composition predominantly takes place by diffusion. However, the transport mechanism is not limited to diffusion, but may in principle be mass transport mechanisms well known in the art, including but not limited to dissolution followed by diffusion across the membrane or diffusion through liquid-filled pores within the membrane. The coating may be non-porous or porous.

As mentioned above, a release-rate modifier is included in the particulate composition. However, such a release-rate modifier may be employed in any possible manner, e.g. in the first composition, in the second composition, added to the second composition after the controlled agglomeration process has ended and, furthermore, a release rate modifier may be present in a coating composition that is applied to the particulate material obtained after controlled agglomeration or it may be applied to a solid dosage form obtained by processing the particulate material obtained after controlled agglomeration into such a dosage form. Thus, dependent on the final presentation of the composition, the specific requirements claimed may be relevant for the particulate composition or the solid dosage form.

Accordingly, different kinds of modified release compositions can be obtained based on a method according to the invention. Without limiting the scope of the invention to the specifically mentioned modified release compositions the following gives a short summary on technologies that may be combined with the controlled agglomeration process in order to achieve a product having the desired properties.

Single Unit Systems

Uncoated tablets: Matrix tablets composed of hydrophilic material: This is one of the most common ways of obtaining a slow release effect. The manufacturing process is quite simple (granulation and compression) and the release controlling ingredients quite cheap and available from many suppliers.

The tablets are normally made from direct compression or simple wet-granulation and then compression. The release-rate modifying substances may be hydrocolloids like: hydroxypropyl methylcellulose (HPMC), sodium carboxymethylcellulose, hydroxypropyl cellulose (HPC), poly (ethylene oxide), poly-vinyl alcohol, poly-vinylpyrollidone, xanthan gum, carbomer, carrageenan, alginates, pectinates, chitosans.

Concentrations and chain lengths, as well as chemical modifications, will determine the release profile. The hydrogel layer that forms when the tablet comes into contact with water brings about the slow release. Release from these types of formulations is dependant on diffusion as well as erosion, normally with a square root dependency, and occasionally zero order dependency.

Matrix tablets composed of hydrophobic material: In this kind of tablets the drug release is retarded through the use of hydrophobic material. Normally, granulation and compression are used. The hydrophobic material can be incorporated via simple dry mixing, melt granulation or through the use of an organic solvent.

Suitable materials are: Different types of methacrylates, mixtures of mono-, di-, and tri-glycerides, oils and waxes, mixtures of polar and non-polar lipids, cellulose derivatives like ethyl cellulose and others.

The release is typically partly diffusion and erosion brought about by enzymatic degradation and peristaltic movements.

Compression coating with hydrophilic material: The release profile resembles the one seen from film-coated tablets. Compression coated tablet normally has a lag-time, and the manufacturing costs are relative high.

The process requires a special compression machine, where an initial tablet, with the drug in, is placed in the die and on a bed of hydrocolloids. On top the other half of the gelling or swelling material is placed and the “tablet” is compressed.

Compression coated tablets from sensitive material: The manufacturing method is similar to the one described above. The release is a typical delayed release. The triggering factor for release could be pH, enzymes or bacteria. The most commonly used delay mechanism is change of pH. pH-sensitive polymers are described herein below.

Coated tablets: Tablets coated with a semi-permeable film: The core tablets can be manufactured in many ways. They can be compressed from simple dry mixtures, from intensive mixer granules, fluid bed granules, and melt granulations.

Coating of the tablets can be done in fluid beds or coating pans. The film material used can be dissolved in organic solvents or made to o/w emulsions. It is normally sprayed onto the moving tablets, in some kind of heated environment. Coating of tablets may be carried out using equipment known in the art. Beads may also be coated using a rotary granulator, such as a CF-granulator available from Freund Corp.

Examples of polymers which provide a semi-permeable membrane are cellulose acetate and cellulose acetate butyrate, and ethylcellulose.

Tablets coated with an impermeable film: The method is the same as above, but the type of film former is different.

Examples of coating materials include film-forming polymers and waxes. Especially suitable for use are thermoplastic polymers, such as poly(ethylene-co-vinyl acetate), poly(vinyl chloride), ethylcellulose, and cellulose acetate, methacrylates and others. These materials may also exhibit the desired low permeation rate of drug, when applied as coatings of thickness greater than about 100 μm.

Release can be created from either drilling a hole in the coating, or suspending water-soluble crystals in the coating.

The release profile from the drilled hole is very accurate (Oros®). The more randomly pin holed film has a more unpredictable drug release.

Tablets coated with sensitive films: These tablets are manufactured like above. The materials used are the same as under “compression coated tablets of sensitive material”.

Multiple-Unit Systems

Uncoated particulates: Particles composed of hydrophobic material: When the diameter of particles becomes less than 300 micron, the delay in release from hydrocolloid matrixes is normally regarded as insignificant and cannot be used effectively. It is however still possible to compose particles of that size from hydrophobic material, and still get a significant delay in release.

The useful materials for that are: Waxes, fats and solid lipids of different kinds. Other hydrophobic materials like magnesium stearate and calcium stearate are also useful. The manufacturing methods for this kind of particulates are several. The granules, beads, or granules are normally manufactured on an intensive mixer, fluid bed, extrusion and spheronisation equipment, spray dryer, or melt granulation equipment.

Ion-exchange resins: Use of ion-exchange resins for linking drug is a feasible way of making delayed release. If the drug is tightly enough bound until it reaches the area of exchange, then this system can work without coating and in the size range of around 300-500 micron.

Not all ion exchange resins can be used, but some are approved for oral use.

Coated particulates: Particles coated with semipermeable film. Even though is requires a great deal of skills, effective controlled release coating of particles down to 100 micron is possible. The resulting particle is normally 100 to 200 micron larger in diameter.

The quality and effectiveness off the coating increases with the roundness of the particles. Elongated particles are much more difficult to coat.

The coating material is identical to the material used in tablet coating. The process in the present invention is suitably fluid bed coating.

The coating is normally chosen from one or more materials selected from the following: shellac; waxes such as, e.g., beeswax, glycowax, castor wax, carnauba wax; hydrogenated oils such as, e.g., hydrogenated castor oil, hydrogenated coconut oil, hydrogenated rape seed oil, hydrogenated soybean oil; fatty acid or fatty alcohol derivatives such as, e.g. stearyl alcohol, glyceryl monostearate, glyceryl distearate, glycerol palmitostearate; acrylic polymers such as, e.g., acrylic resins (Eudragit® RL and RS acrylic resins are copolymers of acrylic and methacrylic acid esters with a low content of quaternary ammonium groups) poly(methyl methacrylate), methacrylate hydrogels, ethylene glycol methacrylate; polylactide derivatives such as, e.g., dl-polylactic acid, polylactic-glycolic acid copolymer; cellulose derivatives, such as, e.g., ethylcellulose, cellulose acetate, cellulose propionate, cellulose butyrate, cellulose valerate, cellulose acetate propionate, cellulose acetate butyrate; vinyl polymers such as, e.g., polyvinyl acetate, polyvinyl formal, polyvinyl butyryl, vinyl chloride-vinyl acetate copolymer, ethylene-vinyl acetate copolymer, vinyl chloridepropylene-vinyl acetate copolymer, polyvinylpyrrolidone; glycols such as, e.g., 1,3-butylene glycol, polyethylene glycols; polyethylene; polyester, polybutadiene; and other high molecular synthetic polymers.

Particles coated with sensitive film: As above just using sensitive film coating as described under tablet.

Some inherent problems with controlled release are often impaired bioavailability (compared to immediate release formulations), risk of dose dumping, large tablets, and non-dividable dosage forms). However, by employment of a method of the present invention it is contemplated that such problems are significantly reduced.

Modified Release Formulation in Combination with Controlled Agglomeration

Controlled agglomeration is a process technique based on spraying an oily material on particles resulting in high load of the oily material. The drug substance may be dissolved or suspended as micronised or nano-sized particles in a melted vehicle (first composition) as a solid dispersion. In case the active substance is poorly water-soluble substance, a bioavailability enhancing effect might be obtained as a result of the solid dispersion obtained. In the second composition the carrier particles are (e.g. lactose) or porous particles (e.g. silicon dioxide) and may include the active substance. The process is normally performed in a fluid bed where the melted oily material is sprayed on cooled or colder particles. However, the process is not restricted to the fluid bed technology, but may be performed in a high shear mixer as well. The product appears as a granular material (multi-particulate system) suitable for direct tablet compression or capsule filling.

The process of controlled agglomeration may be combined with the general modified release formulation principles as described above. Descriptions and examples of the combined process and formulation technology are described in the experimental section.

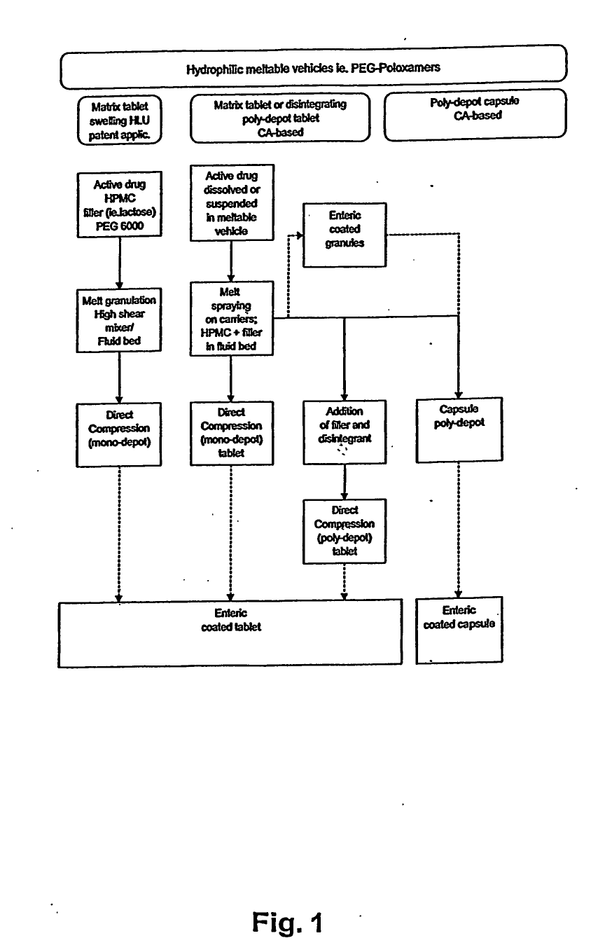

A more general overview is given in FIGS. 1-3.

An example of a matrix tablet or multiparticulate release system based on an intra-granular hydrocolloid phase is outlined in FIG. 1. A matrix modified release tablet or multiparticulate matrix can be obtained by spraying the melted vehicle containing the drug substance on carrier particles comprising a hydrocolloid alone or in combination with a filler like e.g. lactose. As described for controlled agglomeration the drug substance might be dissolved or suspended in the vehicle or being a part of the carrier particles. The outcome of the controlled agglomeration technique in this case is a granular multi-particulate product containing an intragranular hydrocolloid matrix, which could be either directly compressed into a matrix-tablet or followed by addition of proper filler(s) and disintegrate(s) compressed into a tablet which disintegrates into a multi-particulate release system. Alternatively to tablet compression the granular product is filled into hard gelatine capsules. The tablet, capsule or multi-particulate product might be enteric coated to obtain delayed release or it may be coated e.g. to obtain a taste-masking effect, a modified release of the active substance or to facilitate intake of the composition etc.

It is likely that the multi-particulate release principle is more appropriate for poorly soluble drug substance, since a larger surface area is exposed to the dissolution process compared to a matrix tablet. Addition of a hydrocolloid might not be necessary in case the meltable vehicle exhibits gel-forming properties in aqueous environment, (e.g. Poloxamers).

Alternatively to melt spraying a matrix tablet might be based on melt granulation of at mixture of a hydrocolloid, meltable oily material and fillers heating up the mixture during agitation in a high shear mixer or a fluid bed. The outcome is a granular product with a lower content of the oily material compared to controlled agglomeration. The product is suitable for tablet compression into a matrix tablet

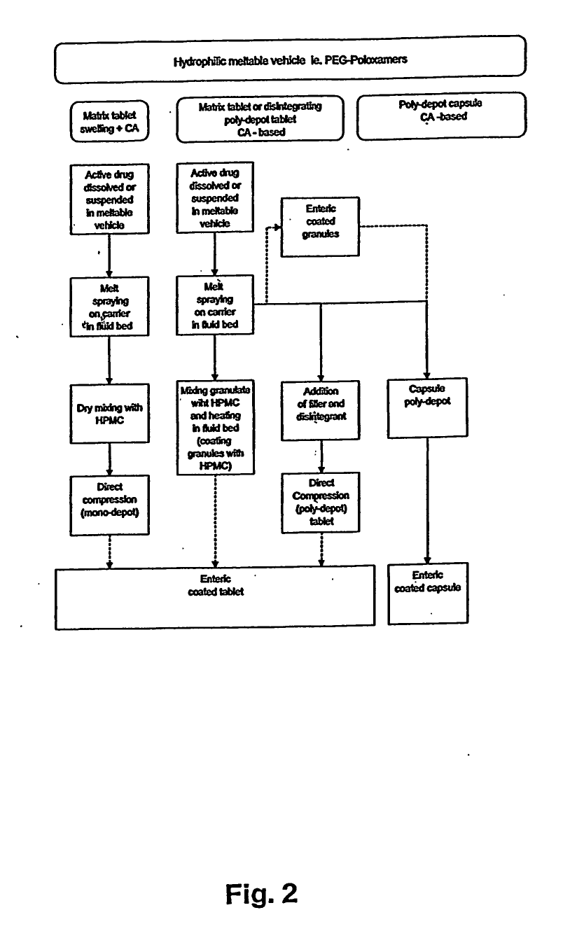

FIG. 2 illustrates a matrix tablet or multi-particulate release system based on an extra-granular hydrocolloid. In this case the swelling hydrocolloid agent is added after the CA-process (controlled agglomeration process) as shown in FIG. 2. Subsequent to the CA process the hydrocolloid might be dry mixed with the granular CA-product (left column in FIG. 2) followed by direct compressed into a matrix tablet. Alternatively, the swelling agent is adhered to the surface of the granular CA-product by heating the product up to close to melting point of the vehicle as shown in FIG. 2 (the two columns to the right). The process requires carefully product temperature control, which is obtainable in a fluid bed. As a result of this process the gel-forming particles are adhered around the CA-granules resulting in a gel-forming release barrier. The granular product is either compressed into a matrix-tablet or into a multi-particulate poly-depot tablet. Capsule filling is an alternative to the poly-depot tablet.

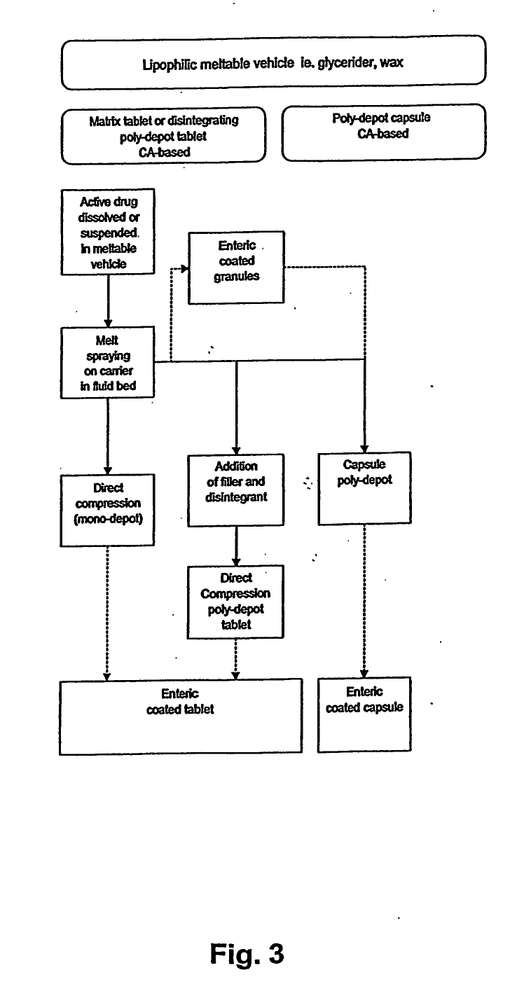

FIG. 3 illustrates matrix tablet or multi-particulate release system based on an intra-granular hydrophobic phase. The previously described modified release systems are based on a gel-forming swelling matrix as drug release control principle using a hydrophilic meltable vehicle as vehicle for the drug substance. Another formulation principle is based on incorporating the drug substance into a hydrophobic or amphiphilic vehicle (e.g. glycerides). FIG. 3 shows the principle of incorporating a lipophilic vehicle by the CA-technique resulting in a matrix tablet or a poly-depot tablet or capsule. The combination of using a lipophilic vehicle and a swelling gel-forming matrix component is optional.

The methods of the invention enable incorporation in a solid material of a high load of an oily material of a type that e.g. due to its solubility properties enables a high load of therapeutically and/or prophylactically active substances with a relatively low aqueous solubility. The oily material is normally solid or semi-solid and normally it has a sticky, oily or waxy character. However, the oily material may also be fluid at room temperature or even at temperature below 5° C. and in such cases it is contemplated that the method may be carried out by employment of cooling of the second composition. By employment of the controlled agglomeration method a particulate material with a high load of carrier may be prepared and the resulting particulate material appears as a particulate powder in solid form. The particulate material obtained by the novel method has excellent properties with respect to flowability, bulk density, compactability and thus, it is suitable for use in the preparation of e.g. tablets. Although the particulate material may have a high load of a carrier of substantially sticky character the particulate material prepared has minimal, if any, adherence to tablet punches and/or dies during manufacture of tablets.

The method according with the invention comprising obtaining a modified release particulate material, wherein the modified release may be obtained by coating with a modified release coating may be performed in a so-called one-pot process, i.e. involving only one type of equipment. To this end, employment of a fluid-bed is excellent.

In the following are described details with respect to the invention and suitable materials for use in the method according to the invention.

First Composition

As mentioned, the method of the invention involves the use of a first composition that is sprayed on a second composition. Normally, the first composition is heated to a suitable temperature and kept at about this temperature while the spraying takes places. In such cases, it is important that the first composition has a viscosity that enables it to be sprayed e.g. by means of a nozzle. Another important issue, when the first composition is heated, is to take the necessary steps to keep the first composition at an elevated temperature e.g. by means of a heating jacket during the application of the first composition to the second composition. However, in those cases where the first composition is in liquid form without any external heating, the heating may be omitted.

The first composition comprises an oily material. Such a material is very important to include in the process as it has been found that the properties of the final composition are markedly improved e.g. with respect to dissolution of an active substance and with respect to bioavailability when a relatively large amount of an oily material is included in this first composition. As it appears from the discussion under the heading “Second composition”, the present inventors have founds materials that are especially suitable as oil sorption materials for use in the present context. A combined use of an oily material (in the first composition) and an oil sorption material (in the second composition) is an especially interesting aspect of the present invention.

In the present context the term “oily materials” is used in a very broad sense including oils, waxes, semi-solid materials and materials that normally are used as solvents (such as organic solvents) or co-solvents within the pharmaceutical industry, and the term also includes therapeutically and/or prophylactically active substances that are in liquid form at ambient temperature; furthermore the term includes emulsions like e.g. microemulsions and nanoemulsions and suspensions. The oily materials that can be absorbed by a material according the invention will normally be liquid at ambient or elevated temperature (for practical reasons the max. temperature is about 25° C.). They may be hydrophilic, lipophilic, hydrophobic and/or amphiphilic materials.

Oily materials suitable for use in the present context are substances or materials having a melting point of at least about 0° C. and at the most about 250° C.

In specific embodiments of the invention, the oily material has a melting point of about 5° C. or more such as, e.g., about 10° C. or more, about 15° C. or more, about 20° C. or more or about 25° C. or more.

In further embodiments of the invention, the oily material has a melting point of at least about 25° C. such as, e.g., at least about 30° C. at least about 35° C. or at least about 40° C. For practical reasons, the melting point may normally not be too high, thus, the oily material normally has a melting point of at the most about 300° C. such as, e.g., at the most about 250° C., at the most about 200° C., at the most about 150° C. or at the most about 100° C. If the melting point is higher a relatively high temperature may promote e.g. oxidation or other kind of degradation of an active substance in those cases where e.g. a therapeutically and/or prophylactically active substance is included.

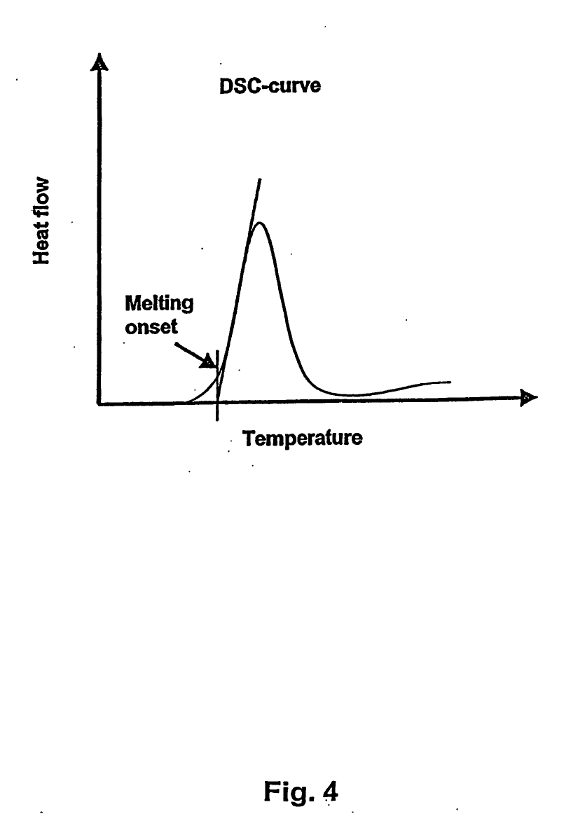

In the present context, the melting point is determined by DSC (Differential Scanning Calorimetry). The melting point is determined as the temperature at which the linear increase of the DSC curve intersects the temperature axis (see FIG. 4 for further details).

Interesting oily materials are generally substances, which are used in the manufacture of pharmaceuticals as so-called melt binders or solid solvents (in the form of solid dosage form), or as co-solvents or ingredients in pharmaceuticals for topical use. It may be hydrophilic, hydrophobic and/or have surface-active properties. In general hydrophilic and/or hydrophobic oils or oily-like materials are suitable for use in the manufacture of a pharmaceutical composition comprising a therapeutically and/or prophylactically active substance that has a relatively low aqueous solubility and/or when the release of the active substance from the pharmaceutical composition is designed to be immediate or non-modified. Hydrophobic oily materials, on the other hand, are normally used in the manufacture of a modified release pharmaceutical composition. The above-given considerations are simplified to illustrate general principles, but there are many cases where other combinations of oils or oily-like materials and other purposes are relevant and, therefore, the examples above should not in any way limit the invention.

Typically, a suitable hydrophilic oily material is selected from the group consisting of: polyether glycols such as, e.g., polyethylene glycols, polypropylene glycols; polyoxyethylenes; polyoxypropylenes; poloxamers and mixtures thereof, or it may be selected from the group consisting of: xylitol, sorbitol, potassium sodium tartrate, sucrose tribehenate, glucose, rhamnose, lactitol, behenic acid, hydroquinon monomethyl ether, sodium acetate, ethyl fumarate, myristic acid, citric acid, Gelucire 50/13, other Gelucire types such as, e.g., Gelucire 44/14 etc., Gelucire 50/10, Gelucire 62/05, Sucro-ester 7, Sucro-ester 11, Sucro-ester 15, maltose, mannitol and mixtures thereof.

A suitable hydrophobic oily material may be selected from the group consisting of: straight chain saturated hydrocarbons, sorbitan esters, paraffins; fats and oils such as e.g., cacao butter, beef tallow, lard, polyether glycol esters; higher fatty acid such as, e.g. stearic acid, myristic acid, palmitic acid, higher alcohols such as, e.g., cetanol, stearyl alcohol, low melting point waxes such as, e.g., glyceryl monostearate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, or a mixture thereof.

In an interesting embodiment, the oily material is a polyethylene glycol having an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In another interesting embodiment, the oily material is polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

In another embodiment, the oily material is a poloxamer such as, e.g. Poloxamer 188, Poloxamer 237, Poloxamer 338 or Poloxamer 407 or other block copolymers of ethylene oxide and propylene oxide such as the Pluronic® and/or Tetronic® series. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

The oily material may also be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

The oily material may of course comprise a mixture of different oils or oily-like materials such as, e.g., a mixture of hydrophilic and/or hydrophobic materials.

Other suitable oily materials may be solvents or semi-solid excipients like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid inter-esterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

In those cases where the first composition contains more than an oily material, the requirements with respect to the melting point mentioned above normally also apply to the first composition, especially in those cases where a minor amount of water is included in the carrier composition. However, when the first composition is heated the first composition may be in the form of two or more phases (e.g. two distinct liquid phases, or a liquid phase comprising e.g. an active substance dispersed therein). In such cases, the melting point is not a true melting point but merely a heating point where the first composition becomes in a liquid form, which is suitable for use in a spraying device. Often such a heating point will for practical purposes correspond to the melting point of the oily material itself.

The total concentration of oily material(s) in the first composition is normally in a range of from about 5 to about 100% w/w such as, e.g., from about 10 to about 99.5% w/w, from about 15 to about 99% w/w, from about 15 to about 98% w/w, from about 15 to about 97% w/w, from about 20 to about 95% w/w such as at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w or at least about 98% w/w.

As explained above, in a process according to the invention the oily material or, the first composition is brought on liquid form by heating to a temperature, which causes the oily material and/or the first composition to melt, and the first composition in liquid form (i.e. as a solution or a dispersion) is sprayed on the second composition.

An important embodiment of the invention includes a method for the preparation of a particulate material, wherein one or more active substances are included in the first composition. In such cases it may be possible to dissolve or finely disperse the active substance in the oily material optionally by heating and thereby apply the active substance to the second composition in a dissolved or finely dispersed state. Thus, the resulting composition may contain the active substance at least partly in a dissolved form (i.e. so-called solid solution or molecular dispersion).

As mentioned above, the first composition in melted or liquidized form is sprayed on a second composition. Thus, the first composition should have a suitable viscosity. If the viscosity is too high, the first composition will be too “thick” and will have a tendency of adhering to the nozzle, which may result in that the delivery through the nozzle is stopped. For the present purpose a viscosity of the first composition is suitably if the viscosity (Brookfield DV-III) is at the most about 800 mPas at a temperature of at the most 100° C. such as, e.g., at the most 700, at the most 600, at the most 500 mPas. In those cases where the melting point of the oily material or the first composition is more than about 80° C., the viscosity values mentioned above are at a temperature of about 40° C. above the melting point.

In the particulate material obtained by a process according to the invention, the concentration of the oily material is from about 5 to about 95% w/w such as, e.g. from about 5 to about 90% w/w, from about 5 to about 85% w/w, from about 5 to about 80% w/w, from about 10 to about 75% w/w, from about 15 to about 75% w/w, from about 20 to abut 75% w/w, from about 25% to about 75% w/w, from about 30% to about 75% w/w. from about 35% to about 75% w/w, from about 25% to about 70% w/w, from about 30% to about 70% w/e, from about 35% to abut 70% w/w. from about 40% to about 70% w/w, from about 45% to about 65% w/w or from about 45% to about 60% w/w.

In those cases where the second composition comprises a pharmaceutically cceptable excipient that has a relatively high particle density it is preferred that the oncentration of the oily material in the particulate material obtained by a process of the invention is from about 5 to about 95% v/v such as, e.g. from about 5 to about 90% v/v, from about 5 to about 85% v/v, from about 5 to about 80% v/v, from about 10 to about 75% v/v from about 15 to about 75% v/v, from about 20 to abut 75% v/v, from about 25% to about 75% v/v, from about 30% to about 75% v/v, from about 35% to about 75% v/v, from about 25% to about 70% v/v, from about 30% to about 70% v/v, from about 35% to abut 70% v/v, from about 40% to about 70% v/v, from about 45% to about 65% v/v or from about 45% to about 60% v/v.

Normally, the first composition does not contain, or only contain a limited amount, of water. One of the advantages with the method is that it is possible to carry it out without any aqueous media so that water-induced or water-based degradation can be avoided for water-sensitive substances. Accordingly, the first composition is essentially non-aqueous and it contains at the most about 20% w/w water such as at the most about 15% w/w, at the most abut 10% w/w, at the most about 5% w/w or at the most about 2.5% w/w.

Apart from the oily material the first composition may contain other ingredients as well such as, e.g., one or more of the pharmaceutically acceptable excipients mentioned below provided that it does not hinder the ability of the first composition to be brought on a sprayable form. Furthermore, one or more one or more release-rate modifiers and/or one or more active substance may be included in the first composition.

In the following are listed a number of suitable release-rate modifiers as well as active substances and pharmaceutically acceptable excipients for use in a method according to the invention. However, the release-rate modifier as well as the active substance and any pharmaceutically acceptable excipient may be present in any of the first and/or second composition or it may be added in a separate process step after the controlled agglomeration of the second composition by means of applying the first composition has taken place. Accordingly, the individual mention of specific substances (release-rate modifiers, active substances and pharmaceutically acceptable excipients) is also relevant for all other steps in the methods of the invention and not limited to use in a first composition.

Release-Rate Modifiers

Such release-rate modifiers may be hydrophilic materials like e.g. cellulose derivatives including hydroxypropyl methylcellulose (HPMC), sodium carboxymethylcellulose, hydroxypropyl cellulose (HPC), methacrylates, poly(ethylene oxide), polyvinyl alcohol, polyvinylpyrrolidone, xanthan gum, carbomer, carrageenan, alginates, pectinates, chitosans etc. or combinations thereof. The release-rate modifiers may also be hydrophobic materials like e.g. sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof. propylene glycol fatty acid esters such as, e.g. propylene glycol monolaurate, propylene glycol ricinoleate and the like, mono- and diglycerides like e.g. glyceryl monooleate, glyceryl dioleae, glyceryl mono- and/or dioleate, glyceryl caprylate, glyceryl caprate etc.; sterol and sterol derivatives; polyethylene glycol sorbitan fatty acid esters (PEG-sorbitan fatty acid esters) such as esters of PEG with the various molecular weights indicated above, and the various Tween 0 series; polyethylene glycol alkyl ethers such as, e.g. PEG oleyl ether and PEG lauryl ether; sugar esters like e.g. sucrose monopalmitate and sucrose monolaurate; polyethylene glycol alkyl phenols like e.g. the Triton O, X or N series; polyoxyethylene-polyoxypropylene block copolymers such as, e.g., the Pluronic series, the Synperonic series, Emkalyx, Lutrol, Supronic etc. polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soy oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids etc.

Other release-rate modifiers for use in the present context include pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Cellulose acetate phthalate (CAP) may be applied to provide delayed release of drug substance until the drug-containing tablet has passed the sensitive duodenal region, that is to delay the release of drug in the gastrointestinal tract until about 15 minutes, and preferably about 30 minutes, after the drug-containing composition has passed from the stomach to the duodenum. The CAP coating solution may also contain one or more plasticizers, such as diethyl phthalate, polyethyleneglycol-400, triacetin, triacetin citrate, propylene glycol, and others as known in the art. Preferred plasticizers are diethyl phthalate and triacetin. The CAP coating formulation may also contain one or more emulsifiers, such as polysorbate-80.

Anionic acrylic copolymers of methacrylic acid and methylmethacrylate are also particularly useful coating materials for delaying the release of drug from the drug-containing composition until the composition has arrived at a position in the small intestine which is distal to the duodenum. Copolymers of this type are available from RohmPharma Corp, under the tradenames Eudragit-L® and Eudragit-S®. Eudragit-L® and Eudragit-S® are anionic copolymers of methacrylic acid and methylmethacrylate. The ratio of free carboxyl groups to the esters is approximately 1:1 in Eudragit-L® and approximately 1:2 in Eudragit-S®. Mixtures of Eudragit-L® and Eudragit-S® may also be used. For coating of drug-containing compositions, these acrylic coating polymers must be dissolved in an organic solvent or mixture of organic solvents. Useful solvents for this purpose are acetone, alcohols like ethanol or isopropyl alcohol, and methylene chloride. It is generally advisable to include 5-20% plasticizer in coating formulations of acrylic copolymers. Useful plasticizers are polyethylene glycols, propylene glycols, diethyl phthalate, dibutyl phthalate, castor oil, and triacetin.

The delay time before release of drug, after the “pH-dependent coated tablet” dosage form has exited the stomach, may be controlled by choice of the relative amounts of Eudragit-L® and Eudragit-S® in the coating, and by choice of the coating thickness. Eudragit-L® films dissolve above pH 6.0, and Eudragit-S® films dissolve above 7.0, and mixtures dissolve at any intermediate pH. Since the pH of the duodenum is approximately 6.0 and the pH of the colon is approximately 7.0, coatings composed of mixtures of Eudragit-L® and Eudragit-S® provide protection of the duodenum from drug. If it is desired to delay release of drug until the drug -containing “pH-dependent coated tablet” has reached the colon, Eudragit-S® may be used as the coating material.

Calcium pectinates are candidates for colon release, as the compounds are degraded by bacteria in the lumen.

In those cases, where a modified release coating is applied, one or more materials selected from the following are suitable: shellac; waxes such as, e.g., beeswax, glycowax, castor wax, carnauba wax; hydrogenated oils such as, e.g., hydrogenated castor oil, hydrogenated coconut oil, hydrogenated rape seed oil, hydrogenated soyabean oil; fatty acid or fatty alcohol derivatives such as, e.g, stearyl alcohol, glyceryl monostearate, glyceryl distearate, glycerol palmitostearate; acrylic polymers such as, e.g., acrylic resins (Eudragit® RL and RS acrylic resins are copolymers of acrylic and methacrylic acid esters with a low content of quaternary ammonium groups) poly(methyl methacrylate), methacrylate hydrogels, ethylene glycol methacrylate; polylactide derivatives such as, e.g., di-polylactic acid, polylactic-glycolic acid copolymer; cellulose derivatives, such as, e.g., ethylcellulose, Surelease®, Aquacoat®, cellulose acetate, cellulose propionate, cellulose butyrate, cellulose valerate, cellulose acetate propionate, cellulose acetate butyrate; vinyl polymers such as, e.g., polyvinyl acetate, polyvinyl formal, polyvinyl butyryl, vinyl chloride-vinyl acetate copolymer, ethylene-vinyl acetate copolymer, vinyl chloride-propylene-vinyl acetate copolymer, polyvinylpyrrolidone; glycols such as, e.g., 1,3-butylene glycol, polyethylene glycols; polyethylene; polyester; polybutadiene; and other high molecular synthetic polymers.

In those cases where one or more release-rate modifying substances are applied in the form of a coating, the amount of each release rate modifying substance applied typically corresponds to from about 0.5-5 mg/cm² of the final composition.

The concentration of the relase-rate modifying substance in the final composition is normally at least about 5% w/w such as, e.g., at least about 10% w/w. The concentration may in certain cases be relatively high such as about 60% or more.

Active Substances

A solid pharmaceutical particulate material or a composition according to the invention comprises a therapeutically, prophylactically and/or diagnostically active substance that is modified released from the particulae material or the composition.

In the present context a therapeutically and/or prophylactically active substance includes any biologically and/or physiologically active substance that has a function on an animal such as, e.g. a mammal like a human. The term includes drug substances, hormones, genes or gene sequences, antigen- comprising material, proteins, peptides, nutrients like e.g. vitamins, minerals, lipids and carbohydrates and mixtures thereof. Thus, the term includes substances that have utility in the treatment and/or preventing of diseases or disorders affecting animals or humans, or in the regulation of any animal or human physiological condition. The term also includes any biologically active substance which, when administered in an effective amount, has an effect on living cells or organisms.

Examples on active substances suitable for use in a particulate material or compositon according to the invention are in principle any active substance such as, e.g. freely water soluble as well as more slightly or insoluble active substances. In specific embodiments, the active substance is a substance that has pharmacokinetic properties that make it a candidate for controlled delivery and/or it may also have biopharmaceutical properties that normally are considered as difficult with respect to controlled release (e.g. poor water-solubility etc.). Accordingly, in specific embodiments, the active substance
i) exhibits a bioavailability of less than about 50% when administered to a subject in the form of plain tablets,
ii) has a water-solubility at room temperature of at the most about 10 mg/ml such as, e.g., at the most about 7.5 mg/ml, at the most about 6 mg/ml, at the most about 5 mg or at the most about 4 mg/ml, at the most about 3 mg/ml such as, e.g., at the most about 2 mg/ml, at the most about 1 mg/ml, at the most about 750 microgram/ml, at the most about 500 μg/ml, at the most about 250 μg/ml, at the most about 100 μg/ml, or at the most about 50 μg/ml, or at the most about 25 μg/ml, or at the most about 20 μg/ml or or at the most about 10 μμg/ml,
iii) exhibits a t_{1/2} in plasma of at the most about 8 hours,
iv) exhibits highly variable bioavailability,
v) is subject to first-pass metabolism,
vi) is subject to degradation in the gastrointestinal tract,
vii) is subject to enzymatic degradation in the stomach, duodenum and/or proximal part of ileum, and/or
viii) is subject to food effect.

A method of the present invention provides a pharmaceutical composition with modified release of one or more active substances. The modified release enables a prolongation in the duration of therapeutic, prophylactic and/or diagnostic effect. Accordingly, a method according to the present invention provides a pharmaceutical composition comprising a sufficient amount of at least one release-rate modifier so that following ingestion by a subject in need thereof the active substance is released in the gastrointestinal tract of the mammal at a rate so that less than 85% w/w is released within the first 30 min after ingestion.

In specific embodiments less than about 80% w/w such as, e.g., less than about 75% w/w, less than about 70% w/w, less than about 65% w/w, less than about 60% w/w, less than about 55% w/w, less than about 50% w/w, less than about 45% w/w, less than about 40% w/w, less than about 35% w/w, less than about 30% w/w or less than about 25% w/w is released within about 30 min after ingestion, and/or

less than 85% w/w is released within the first hours, within about 2 hours, within about 3 hours, within about 4 hours, within about 5 hours or within about 6 hours after ingestion, and/or

less than 80% w/w is released such as, e.g., less than about 75% w/w, less than about 70% w/w, less than about 65% w/w, less than about 60% w/w, less than about 55% w/w, less than about 50% w/w or less than about 45% w/w is released within the first hour after ingestion, and/or

less than 80% w/w is released such as, e.g., less than about 75% w/w, less than about 70% w/w, less than about 65% w/w, less than about 60% w/w, less than about 55% w/w or less than about 50% w/w is released within 2 hours after ingestion, and/or

less than 80% w/w is released such as, e.g., less than about 75% w/w, less than about 70% w/w, less than about 65% w/w, less than about 60% w/w, less than about 55% w/w or less than about 50% w/w is released within 3 hours after ingestion, and/or

less than 80% w/w is released such as, e.g., less than about 75% w/w, less than about 70% w/w, less than about 65% w/w or less than about 60% w/w is released within 6 hours after ingestion, and/or

less than 75% w/w is released within about 7 hours, within about 8 hours, within about 9 hours, within about 10 hours, within about 11 hours or within about 12 hours after ingestion, and/or

less than 70% w/w or less than about 65% w/w is released within about 7 hours, within about 8 hours, within about 9 hours, within about 10 hours, within about 11 hours or within about 12 hours after ingestion, and/or

more than 20% w/w such as, e.g., more than about 25% w/w, more than about 30% w/w, more than about 35% w/w or more than about 40% w/w is released within about 7 hours, within about 8 hours, within about 9 hours, within about 10 hours, within about 11 hours or within about 12 hours after ingestion, and/or

more than 20% w/w such as, e.g., more than about 25% w/w, more than about 30% w/w, more than about 35% w/w, more than about 40% w/w, more than about 45% w/w, more than about 50% w/w, more than about 55% w/w or more than about 60% w/w is released within about 15 hours, within about 20 hours or within about 24 hours after ingestion.

As mentioned hereinbefore, the present invention is especially suitable for use to obtain useful modified release composition of active substances that normally are considered to be candidates for modified release due to their pharmacokinetic properties but not due their biopharmaceutical properties. Accordingly, in a specific embodiment the invention related to a method for the preparation of a pharmaceutical composition, wherein the bioavailability (measured as AUC₀) of the active substance after oral administration of the pharmaceutical composition to a subject is at least about 50% such as, e.g., at least about 55%, at least about 60%, at least about 65%, at least about 70%, at least about 75%, at least about 80%, at least about 85%, at least about 90% compared to the bioavailability of the active substance after oral administration of a particulate composition obtained in analogues matter but without any release-rate modifying substances.

Moreover, in a specific embodiment, the invention relates to a method for the preparation of a pharmaceutical composition comprising a sufficient amount of at least one release-rate modifier to provide a modified release of the active substance sufficient to provide a dissolution rate in vitro of the particulate composition, which—when measured according to USP dissolution test (paddle) employing water as dissolution medium, 100 rpm and a temperature of about 37° C. permits release of less than 85% w/w within about 30 min after start of the test. For certain types of compositions, i.e. those provided with an enteric coating or otherwise formulated in order to achieve a pH dependent release, another medium than water may be employed such as, e.g., a buffer having a suitable pH or a combination of dissolution media e.g. as described in USP. A person skilled in the art of testing of pharmaceutical composition will know how to adjust the method to individual situations.

In further embodiments less than about 80% w/w such as, e.g., less than about 75% w/w, less than about 70% w/w, less than about 65% w/w, less than about 60% w/w, less than about 55% w/w, less than about 50% w/w, less than about 45% w/w, less than about 40% w/w, less than about 35% w/w, less than about 30% w/w or less than about 25% w/w is released within about 30 min after start of the test, and/or

less than 85% w/w is released within the first hours, within about 2 hours, within about 3 hours, within about 4 hours, within about 5 hours or within about 6 hours after start of the test, and/or

less than 80% w/w is released such as, e.g., less than about 75% w/w, less than about 70% w/w, less than about 65% w/w, less than about 60% w/w, less than about 55% w/w, less than about 50% w/w or less than about 45% w/w is released within the first hour after start of the test, and/or

less than 80% w/w is released such as, e.g., less than about 75% w/w, less than about 70% w/w, less than about 65% w/w, less than about 60% w/w, less than about 55% w/w or less than about 50% w/w is released within 2 hours after start of the test, and/or

less than 80% w/w is released such as, e.g., less than about 75% w/w, less than about 70% w/w, less than about 65% w/w, less than about 60% w/w, less than about 55% w/w or less than about 50% w/w is released within 3 hours after start of the test, and/or

less than 80% w/w is released such as, e.g., less than about 75% w/w, less than about 70% w/w, less than about 65% w/w or less than about 60% w/w is released within 6 hours after start of the test, and/or

less than 75% w/w is released within about 7 hours, within about 8 hours, within about 9 hours, within about 10 hours, within about 11 hours or within about 12 hours after start of the test, and/or

less than 70% w/w or less than about 65% w/w is released within about 7 hours, within about 8 hours, within about 9 hours, within about 10 hours, within about 11 hours or within about 12 hours after start of the test, and/or

more than 20% w/w such as, e.g., more than about 25% w/w, more than about 30% w/w, more than about 35% w/w or more than about 40% w/w is released within about 7 hours, within about 8 hours, within about 9 hours, within about 10 hours, within about 11 hours or within about 12 hours after start of the test, and/or

more than 20% w/w such as, e.g., more than about 25% w/w, more than about 30% w/w, more than about 35% w/w, more than about 40% w/w, more than about 45% w/w, more than about 50% w/w, more than about 55% w/w or more than about 60% w/w is released within about 15 hours, within about 20 hours or within about 24 hours after start of the test.

Examples on active substances suitable for use are e.g. antibacterial substances, antihistamines and decongestants, anti-inflammatory agents, anti-parasitics, antivirals, local anesthetics, anti-fungals, amoebicidals or trichomonocidal agents, analgesics, anti-anxiety agents, anti-clotting agents, anti-arthritics, anti-asthmatics, anticoagulants, anticonvulsants, antidepressants, antidiabetics, antiglaucoma agents, anti-malarials, antimicrobials, anti-neoplastics, anti-obesity agents, anti-psychotics, anti-hypertensiva, antitussiva, auto-immune disorder agents, anti-impotence agents, anti-Parkinsonism agents, anti-Alzheimers' agents, antipyretics, anti-cholinergics, anti-ulcer agents, anorexic, beta-blockers, beta-2 agonists, beta agonists, blood glucose-lowering agents, bronchodilators, agents with effect on the central nervous system, cardiovascular agents, cognitive enhancers, contraceptives, cholesterol-reducing agents, cytostatics, diuretics, germicidals, H-2 blockers, hormonal agents, hypnotic agents, inotropics, muscle relaxants, muscle contractants, physic energizers, sedatives, sympathomimetics, vasodilators, vasoconstrictors, tranquilizers, electrolyte supplements, vitamins, counterirritants, stimulants, anti-hormones, drug antagonists, lipid-regulating agents, uricosurics, cardiac glycosides, expectorants, purgatives, contrast materials, radiopharmaceuticals, imaging agents, peptides, enzymes, growth factors, etc. Specific examples include e.g. aAnti-inflammatory drugs like e.g. ibuprofen, indometacin, naproxen, nalophine; aAnti-Parkinsonism agents like e.g. bromocriptine, biperidin, benzhexol, benztropine etc.; antidepressants like e.g. imipramine, nortriptyline, pritiptyline, etc.; antibiotics like e.g. clindamycin, erythomycin, fusidic acid, gentamicin, mupirocine, amfomycin, neomycin, metronidazol, sulphamethizole, bacitracin, framycetin, polymyxin B, acitromycin etc.; antifungal agents like e.g. miconazol, ketoconaxole, clotrimazole, amphotericin B, nystatin, mepyramin, econazol, fluconazol, flucytocine, griseofulvin, bifonazole, amorofine, mycostatin, itraconazole, terbenafine, terconazole, tolnaftate etc.; antimicrobial agents like e.g.metronidazole, tetracyclines, oxytetracylines, peniciilins etc.; antiemetics like e.g. metoclopramide, droperidol, haloperidol, promethazine etc.; antihistamines like e.g. chlorpheniramine, terfenadine, triprolidine etc.; antimigraine agents like e.g. dihydroergotamine, ergotamine, pizofylline etc.; coronary, cerebral or peripheral vasodilators like e.g. nifedipine, diltiazem etc.; antianginals such as, e.g., glyceryl nitrate, isosorbide dinitrate, molsidomine, verapamil etc.; calcium channel blockers like e.g. verapamil, nifedipine, diltiazem, nicardipine etc.; hormonal agents like e.g. estradiol, estron, estriol, polyestradiol, polyestriol, dienestrol, diethylstilbestrol, progesterone, dihydroprogesterone, cyprosterone, danazol, testosterone etc.; contraceptive agents like e.g. ethinyl estradiol, lynestrenol, etynodiol, norethisterone, mestranol, norgestrel, levonorgestrel, desodestrel, medroxyprogesterone etc.; antithrombotic agents like e.g. heparin, warfarin etc.; diuretics like e.g. hydrochlorothiazide, flunarizine, minoxidil etc.; antihypertensive agents like e.g. propanolol, metoprolol, clonidine, pindolol etc.; corticosteroids like e.g. beclomethasone, betamethasone, betamethasone-17-valerate, betamethasone-dipropionate, clobetasol, clobetasol-17-butyrate, clobetasol-propionate, desonide, desoxymethasone, dexamethasone, diflucortolone, flumethasone, flumethasone-pivalte, fluocinolone acetonide, fluocinoide, hydrocortisone, hydrocortisone-17-butyrate, hydrocortisone-buteprate, methylprednisolone, triamcinolone acetonide, hacinonide, fluprednide acetate, alklometasone-dipropionate, fluocortolone, fluticason-propionte, mometasone-furate, desoxymethasone, diflurason-diacetate, halquinol, cliochinol, chlorchinaldol, fluocinolone-acetonide etc.; dermatological agents like e.g. nitrofurantoin, dithranol, clioquinol, hydroxyquinoline, isotretionin, methoxsalen, methotrexate, tretionin, trioxalen, salicylic acid, penicillamine etc.; steroids like e.g. estradiol, progesterone, norethindrone, levonorgestrel, ethynodiol, levonorgestrol, norgestimate, gestanin, desogestrel, 3-keton-desogesterel, demegestone, promethoestrol, testosterone, spironolactone and esters thereof etc.; nitro compounds like e.g. amyl nitrates, nitroglycerine and isosorbide nitrate etc.; opioids like e.g. morphine, buprenorphine, oxymorphone, hydromorphone, codeine, tramadol etc.; prostaglandins such as, e.g., a member of the PGA, PGB, PGE or PGF series such as, e.g. minoprostol, dinoproston, carboprost, eneprostil etc.; peptides like e.g. growth hormone releasing factors, growth factors (e.g. epidermal growth factor (EGF), nerve growth factor (NGF), TGF, PDGF, insulin growth factor (IGF), fibroblast growth factor (aFGF, bFGF etc.), somatostatin, calcitonin, insulin, vasopressin, interferons, IL-2 etc., urokinase, serratiopeptidase, superoxide dismutase, thyrotropin releasing hormone, lutenizing hormone releasing hormone (LH-RH), corticotrophin releasing hormone, growth hormone releasing hormone (GHRH), oxytocin, erythropoietin (EPO), colony stimulating factor (CSF) etc.

Interesting examples are also prescription drugs like:
Cardiovascular drugs: Zocor®, Lipitor®, Prevachol®, Mevalotin®, Mevacor®, Lescol®, TriCor®, Norvasc®, Cozaar and Hyzaar®, Prinivil and Prinzide®, Diovan®/Co-Diovan®, Zestril®, Vasotech® and Vaseretic®, Lotensin®/Cibacen® and Lotrel®, Adalat®, Toprol-XL®/Seloken®, Tritace®/Delix®, Accupril® and Accuretic®, Avapro® and Avalide®, Plendil®, Monopril®, Blopress®, Atacand®, Tenormin®, Avapro®/Aprovel®, Coreg®, Altace®, Capoten®, Plavix®, Lovenox®/Clexane®, Fraxiparine®, ReoPro®, Panaldine®, Cordarone®,
Central nervous system drugs: Paxil/Seroxat®, Zolotoft®, Prozac®, Prozac Weekly® and Sarafem®, Effexor®, Wellbutrin®, Celexa®, Remeron®, Serzone®, Zyprexa®, Risperdal®, Seroquel®, Clozarill®/Leponex®, Neurontin®, Depaktoke®, Lamictal® Topamax®, Tegretol®, Imitrex®/Imigran®, Zomig®, Maxalt®, Amblen®, Stilnox®, Ultane®/Sevorane®, Diprivan®, BuSpar®, Xanax®, Aricept®, Memantine®, Adderall®, Dystonia®, Botox®,
Anti-infective agents: Augmentin®, Cipro®/Ciprobay®, Zithromax®, Biaxin®, Levaquin® and Floxin®, Rocephin®, Primaxin®, Ceftin®/Zinnat®, Cravit®, Zosyn®/Tazocin®, Cefzil®, Tequin®, Tortaz®/Fortum®, Combivir®, Zerit®, Valtrex®, Epivir®, Zovirax®, Crixivan®, Viracept®, Viramune®, Kaletra®, Diflucan®, Lamisil®, Sporanox®,
Respiratory drugs: ClaritinAllegra® Telfast®, Zyrtec®, Flonase®/Flixonase®, Atrovent®, Nasonex®, Rhinocort®, Alesion®, Singulair®, Flovent®/Flixotide®, Advair®/Seretide®, Serevent®, Pulmicort®, Ventoline®, Combivent®, Synagis®, Mucosolvan®,
Gastrointestinal drugs: Prilosec®/Losec®, Prevacid®, Gaster®, Takepron®, Zantac®, Pantozol Nexium Protonix®, Aciphex®/Pariet®, Pepcid®, Axid®, Zoton®, Zofran®,
Cancer drugs: Taxol®, Taxotere®, Nolvadex®, Herceptin Ellence®/Pharmorubicin®, Lupron®, Zoladex®, Leuplin®, Casodex®, Intron A®, Peg-Intron® and Rebertron®, Rituxan®, Gemzar®, Paraplatin®, Camptosar®,
Antiarthritic drugs/analgesics: , Celebrex®, Vioxx®, Enbrel®, Remicade®, Voltaren®, Mobic®, Duragesic®, Ultram® and Ultrcet®,
Blood disorder treatments: Procrit®/Eprex®, Epogen®, Epogin®, NeoRecormon®, Neupogen®, NovoSeven®,
Diabetes drugs: Glucophage®, Humulin Avandia®, Humalog®, Actos®, Amaryl®,
Glucovance®, Glucophage XR®, Glucotrol XL®, Precose®/Glucobay®,
Bone metabolism regulators: Fosamax®, Evista®, Miacalcin®, Actone®, Aredia®,
Urinary disorder agents: Harnal®, Proscar®, Cardura®, Flomax®, Detrol®,
Hormones: Premarin®, Premphase® And prempro®, Estraderm®, Synthroid®,
Immunosuppressive agents: Neoral®/Sandimmun®, CellCept®, Rapamune®, Prograf®, Medrol®,
Multiple Sclerosis drugs: Avonex®, Betaseron®/Betaferon®, Rebif®, Copaxone®,
Biologicals: Prevnar®, Engerix-B®, Infanrix®, Gamimune N®,
Sexual dysfunction drugs: Viagra®,
Imaging agents: lopamiron®, Omnipaque®, Magnevist®,
Ophthalmic drugs: Xalatan®, Trusopt® and Cosopt®,
Dermatological drugs: Accutane®/Roaccutan®, Cleocin®,
Growth failure therapies: Genotropin®, Humatrope®,
Infertility drugs: Gonal-F®, Follistim(Puregon®),
Gaucher disease drugs: Cerezyme®,
Obesity drugs: Xencial®,
Acromegaly drugs: Sandostatin®,
Contraceptives: Depo-Provera®,

Other interesting examples of active substances that are slightly soluble, sparingly soluble or insoluble in water are given in Table 1 and 2 below:

TABLE 1
Poorly-Soluble Drug Candidates

| Drug Name | Therapeutic Class | Solubility in water |
|---|---|---|
| Alprazolam | CNS | Insoluble |
| Amiodarone | Cardiovascular | Very Slightly |
| Amlodipine | Cardiovascular | Slightly |
| Astemizole | Respiratory | Insoluble |
| Atenolol | Cardiovascular | Slightly |
| Azathioprine | Anticancer | Insoluble |
| Azelastine | Respiratory | Insoluble |
| Beclomethasone | Respiratory | Insoluble |
| Budesonide | Respiratory | Sparingly |
| Buprenorphine | CNS | Slightly |
| Butalbital | CNS | Insoluble |
| Carbamazepine | CNS | Insoluble |
| Carbidopa | CNS | Slightly |
| Cefotaxime | Anti-infective | Sparingly |
| Cephalexin | Anti-infective | Slightly |
| Cholestyramine | Cardiovascular | Insoluble |
| Ciprofloxacin | Anti-infective | Insoluble |
| Cisapride | Gastrointestinal | Insoluble |
| Cisplatin | Anticancer | Slightly |
| Clarithromycin | Anti-infective | Insoluble |
| Clonazepam | CNS | Slightly |
| Clozapine | CNS | Slightly |
| Cyclosporin | Immunosuppressant | Practically Insoluble |
| Diazepam | CNS | Slightly |
| Diclofenac sodium | NSAID | Sparingly |
| Digoxin | Cardiovascular | Insoluble |
| Dipyridamole | Cardiovascular | Slightly |
| Divalproex | CNS | Slightly |
| Dobutamine | Cardiovascular | Sparingly |
| Doxazosin | Cardiovascular | Slightly |
| Enalapril | Cardiovascular | Sparingly |
| Estradiol | Hormone | Insoluble |
| Etodolac | NSAID | Insoluble |
| Etoposide | Anticancer | Very Slightly |
| Famotidine | Gastrointestinal | Slightly |
| Felodipine | Cardiovascular | Insoluble |
| Fentanyl citrate | CNS | Sparingly |
| Fexofenadine | Respiratory | Slightly |
| Finasteride | Genito-urinary | Insoluble |
| Fluconazole | Antifungal | Slightly |
| Flunosolide | Respiratory | Insoluble |
| Flurbiprofen | NSAID | Slightly |
| Fluvoxamine | CNS | Sparingly |
| Furosemide | Cardiovascular | Insoluble |
| Glipizide | Metabolic | Insoluble |
| Glyburide | Metabolic | Sparingly |
| Ibuprofen | NSAID | Insoluble |
| Isosorbide dinitrate | Cardiovascular | Sparingly |
| Isotretinoin | Dermatological | Insoluble |
| Isradipine | Cardiovascular | Insoluble |
| Itraconzole | Antifungal | Insoluble |
| Ketoconazole | Antifungal | Insoluble |
| Ketoprofen | NSAID | Slightly |
| Lamotrigine | CNS | Slightly |
| Lansoprazole | Gastrointestinal | Insoluble |
| Loperamide | Gastrointestinal | Slightly |
| Loratadine | Respiratory | Insoluble |
| Lorazepam | CNS | Insoluble |
| Lovastatin | Cardiovascular | Insoluble |
| Medroxyprogesteron | Hormone | Insoluble |
| Mefenamic acid | Analgesic | Slightly |
| Methylprednisolone | Steroid | Insoluble |
| Midazolam | Anesthesia | Insoluble |
| Mometasone | Steroid | Insoluble |
| Nabumetone | NSAID | Insoluble |
| Naproxen | NSAID | Insoluble |
| Nicergoline | CNS | Insoluble |
| Nifedipine | Cardiovascular | Practically Insoluble |
| Norfloxacin | Anti-infective | Slightly |
| Omeprazole | Gastrointestinal | Slightly |
| Paclitaxel | Anticancer | Insoluble |
| Phenytoin | CNS | Insoluble |
| Piroxicam | NSAID | Sparingly |
| Quinapril | Cardiovascular | Insoluble |
| Ramipril | Cardiovascular | Insoluble |
| Risperidone | CNS | Insoluble |
| Saquinavir | Protease inhibitor | Practically insoluble |
| Sertraline | CNS | Slightly |
| Simvastatin | Cardiovascular | Insoluble |
| Terbinafine | Antifungal | Slightly |
| Terfenadine | Respiratory | Slightly |
| Triamcinolone | Steroid | Insoluble |
| Valproic acid | CNS | Slightly |
| Zolpidem | CNS | Sparingly |

TABLE 2
Poorly-Soluble Drugs with Low Bioavailability

| | | Solubility | |
|---|---|---|---|
| Drug Name | Indication | In Water | Bioavailability |
| Astemizole | Allergic Rhinitis | Insoluble | Low-moderate |
| Cyclandelate | Peripheral | Insoluble | Low |
| | vascular dis. | | |
| Perphenazine | Psychotic disorder | Insoluble | Low |
| Testosterone | Androgen Repl. | Insoluble | Low |
| | Therapy | | |
| Famotidine | GERD | Slightly soluble | Low (39-50%) |
| Budesonide | Allergic Rhinitis | Sparingly soluble | Low (˜15%) |
| Mesalamine | Irritable Bowel | Slightly soluble | Low (˜20%) |
| | Syndrome | | |
| Clemastine | Allergic Rhinitis | Slightly soluble | Low (˜39%) |
| fumarate | | | |
| Buprenorphine | Pain | Slightly soluble | Low (<30%) |
| Sertraline | Anxiety | Slightly soluble | Low (<44%) |
| Auranofin | Arthritis | Slightly soluble | Low (15-25%) |
| Felodipine | Hypertension | Insoluble | Low (15%) |
| Isradipine | Hypertension | Insoluble | Low (15-24%) |
| Danazol | Endometriosis | Insoluble | Low |
| Loratadine | Allergic Rhinitis | Insoluble | Low |
| Isosorbide | Angina | Sparingly soluble | Low (20-35%) |
| dinitrate | | | |
| Fluphenazine | Psychotic disorder | Insoluble | Low (2-3%) |
| Spironolactone | Hypertension, | Insoluble | Low (25%) |
| | Edema | | |
| Biperiden | Parkinson's disease | Sparingly soluble | Low (29-33%) |
| Cyclosporin | Transplantation | Slightly soluble | Low (30%) |
| Norfloxacin | Bacterial Infection | Slightly soluble | Low (30-40%) |
| Cisapride | GERD | Insoluble | Low (35-40%) |
| Nabumetone | Arthritis | Insoluble | Low (35%) |
| Dronabinol | ANTIEMETIC | Insoluble | Low 10-20%) |
| Lovastatin | Hyperlipidemia | Insoluble | Low (˜5%) |
| Simvastatin | Hyperlipidemia | Insoluble | Low (<5%) |

The amount of active substance incorporated in a particulate material (and/or in a pharmaceutical, cosmetic or foodstuff composition) may be selected according to known principles of pharmaceutical formulation. In general, the dosage of the active substance present in a particulate material according to the invention depends inter alia on the specific drug substance, the age and condition of the patient and of the disease to be treated.

A particulate material or composition according to the invention may comprise a cosmetically active ingredient and/or a food ingredient. Specific examples include vitamins, minerals, vegetable oils, hydrogenated vegetable oils, etc.

Second Composition

As mentioned above the first composition comprising an oily material is sprayed on a second composition. In order to be able to achieve a high amount of the oily material in the final particulate material and in order to enable a controlled agglomeration of the particles comprised in the second composition, the present inventors have earlier found (WO 03/004001) that in specific embodiments, the second composition should initially have a temperature which is at least about 10° C. such as, e.g., at least about 15° C., at least about 20° C., at least about 25° C., or at least about 30° C. below the melting point of the oily material or the first composition (or the heating point of the carrier composition). However, as mentioned above, a temperature difference of at least about 10° C. it is not always necessary. Thus, the second composition may have a temperature of at the most a temperature corresponding to the melting point of the oily material and/or of the first composition such as, e.g., a temperature of at least about 2° C., at least about 5° C. No external heating of the second composition is normally employed during the controlled agglomeration process, but in some cases it may be advantageous to employ a cooling via the inlet air. However, the temperature of the second composition may increase to a minor extent due to the working of the composition. However, the temperature must (or will) not be higher than at the most the melting point of the oily material or first composition such as, e.g. at the most about 5° C. such as at the most about 10° C., at the most about 15° C. or at the most about 20° C. below the melting point of the oily material or the first composition. Accordingly, a process of the invention can be carried out without any heating of the second composition, i.e. it can be carried out at ambient or room temperature (i.e. normally in a range of from about 20° C. to about 25° C.).

In contrast thereto, known melt granulation methods involve external heating of the material that is to be granulated (or agglomerated) together with a melt binder.

The second composition comprises one or more pharmaceutically and/or cosmetically acceptable excipients and, furthermore, a therapeutically and/or prophylactically active substance may be present in the second composition.

In the present context the terms “pharmaceutically acceptable excipient” and “cosmetically acceptable excipient” are intended to denote any material, which is inert in the sense that it substantially does not have any therapeutic and/or prophylactic effect per se. Such an excipient may be added with the purpose of making it possible to obtain a pharmaceutical and/or cosmetic composition, which has acceptable technical properties.

Examples of suitable excipients for use in a second composition include fillers, diluents, disintegrants, binders, lubricants etc. or mixture thereof. As the particulate material obtained by a process according to the invention may be used for different purposes, the choice of excipients is normally made taken such different uses into considerations. Other pharmaceutically acceptable excipients for use in a second composition (and/or in the carrier composition) are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples on suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel Al 5C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the second composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in the second composition (and/or in the carrier composition) are e.g. colouring agents, taste-masking agents, pH-adjusting agents, solubilizing agents, stabilising agents, wetting agents, surface active agents, antioxidants, agents for modified release etc.

Sorption Materials

In specific embodiments of the invention, the second composition comprises a sorption material for oily materials or such a material may be added after the controlled agglomeration process. Such a sorption material
i) has an oil threshold value of 10% or more, when tested according to the Threshold Test herein, and
at fulfills at least one of the test ii) or iii):
ii) releases at least 30% of an oil, when tested according to the Release Test herein, and
iii) in the form of a tablet has a disintegration time of at the most 1 hour, when tested according to Ph. Eur. Disintegration test, the tablet containing about 90% w/w or more of the pharmaceutically acceptable material.

In the following such a sorption material for oils or oily-like materials is also denoted “oil sorption material”. Furthermore, in the present context the term “sorption” is used to denote “absorption” as well as “adsorption”. It should be understood that whenever one of the terms is used it is intended to cover the phenomenon absorption as well as adsorption.

As it appears from the above, it is important that the oil sorption material fulfils at least two tests. One of the tests is mandatory, i.e. the Threshold Test must be met. This test gives a measure for how much oily material the oil sorption material is able to absorb while retaining suitable flowability properties. It is important that an oil sorption material according to the invention (with or without oil absorbed) has a suitable flowability so that it easily can be admixed with other excipients and/or further processed into compositions without significant problems relating to e.g. adherence to the apparatus involved. The test is described in the Experimental section herein and guidance is given for how the test is carried out. The Threshold Test involves the determination of the flowability of the solid material loaded with different amounts of oil.

From above it is seen that the oil threshold value normally must exceed 10% and often the oil sorption material has an oil threshold value of at least about 15%, such as, e.g., at least about 20%, at least about 25%, at least about 30%, at least about 35%, at least about 40%, or at least about 45%.

A especially suitable oil sorption material for use in a method according to the invention is Aeroperl 300 that has a very high oil threshold value of about 60%. Accordingly, materials that have an oil threshold value of at least about 50%, such as, e.g., at least about 55% or at least about 60% are specifically useful in a method of the present invention.

Furthermore, an oil sorption material for use according to the invention must fulfil at least one further test, namely a release test and/or a disintegration test.

The release test gives a measure of the ability of an oil sorption material to release the oil that is absorbed to the material when contacted with water. This ability is very important especially in those situations where an active substance is contained in the oily material. If the oil sorption material is not capable of releasing the oil from the material then there is a major risk that the active substance will only to a minor degree be released from the material. Accordingly, it is envisaged that bioavailability problems relating to e.g. poor absorption etc. will occur in such situations.

The requirements for the release test are that the sorption material—when tested as described herein—
ii) releases at least about 30% such as, e.g., at least about 35%, at least about 40%, at least about 45%, at least about 50%, at least about 55% or at least about 60% of an oil. As it appears from the examples herein a suitable oil sorption material like Aeroperl 300 has a much higher release. Therefore, in a specific embodiment of the invention, the sorption material—when tested as described herein—
ii) releases at least about 65% such as, e.g., at least about 70%, at least about 75% or at least about 80% of an oil.

The second of the tests at least one of which an oil sorption material according to the invention must fulfill is a disintegration test. The test is not performed on the solid material in particular form but on a tablet made of the solid material. A requirement with respect to disintegration is important in order to ensure that the solid material—when included in solid dosage forms—does not impart unwanted properties to the dosage form e.g. leading to unwanted properties with respect to dissolution and bioavailability of the active substance contained in the dosage form. For some of the materials suitable for use according to the invention it is possible to press tablets containing 100% w/w of the solid material itself. If this is the case, the test is carried out on such tablets. However, it is envisaged that there may be situations where it is rather difficult to prepare tablets from the solid material alone. In such cases it is possible to add pharmaceutically acceptable excipients normally used in the preparation of compressed tablets up to a concentration of 10% w/w or less. Examples on suitable pharmaceutically acceptable excipients include fillers, diluents, binders and lubricants. However, excipients, normally classified as disintegrants, should be avoided.

Accordingly, the sorption material for use according to invention- when tested as described herein
iii) in the form of a tablet should have a disintegration time of at the most 1 hour, when tested according to Ph. Eur. Disintegration test, the tablet containing about 90% w/w or more, such as, e.g., about 92.5% w/w or more, about 95% w/w or more, about 97.5% w/w or more or about 100% of the pharmaceutically acceptable material.

In a further embodiment, the sorption material—when tested as described herein
iii) in the form of a tablet has a disintegration time of at the most about 50 min, such as, e.g., at the most about 40 min, at the most about 30 min, at the most about 20 min, at the most about 10 min or at the most about 5 min, when tested according to Ph. Eur. Disintegration test, the tablet containing about 90% w/w or more, such as, e.g., about 92.5% w/w or more, about 95% w/w or more, about 97.5% w/w or more or about 100% of the pharmaceutically acceptable material.

In a specific embodiment, the oil sorption material fulfils all three tests. Thus, the oil sorption material—when tested as described herein—
i) has an oil threshold value of at least about 10%, such as, e.g., at least about 15%, at least about 20%, at least about 25%, at least about 30%, at least about 35%, at least about 40%, at least about 45%, at least about 50%, at least about 55% or at least about 60%,
ii) releases at least about 30% such as, e.g., at least about 35%, at least about 40%, at least about 45%, at least about 50%, at least about 55%, at least about 60%, at least about 65%, at least about 70%, at least about 75% or at least about 80% of an oil, and
iii) in the form of a tablet has a disintegration time of at the most 1 hour such as at the most about 50 min, at the most about 40 min, at the most about 30 min, at the most about 20 min, at the most about 10 min or at the most about 5 min, when tested according to Ph. Eur. Disintegration test, the tablet containing about 90% w/w or more, such as, e.g., about 92.5% w/w or more, about 95% w/w or more, about 97.5% w/w or more or about 100% of the oil sorption material.

Other specific oil sorption materials for use according to the invention are those, wherein the oil sorption material—when tested as described herein—
i) has an oil treshold value of at least about 55%; the oil sorption material—when tested as described herein—
ii) releases at least about 75% of an oil; and/or the oil sorption material—when tested as described herein—
iii) in the form of a tablet has disintegration time of at the most about 10 min, when tested according to Ph. Eur. Disintegration test, the tablet containing about 97.5% w/w of the oil sorption material.

The oil sorption material for use according to the invention is normally a particulate material in the form of e.g. powders, particles, granules, granulates etc.

Such particulate material that is suitable for use as an oil sorption material according to the invention has normally a bulk density of about 0.15 g/cm³ or more such as, e.g., at least about 0.20 g/cm³ or at least about 0.25 g/cm³.

Furthermore, the oil sorption material normally has an oil absorption value of at least about 100 g oil/100 g such as, e.g., at least about 150 g oil/100 g, at least about 200 g oil/100 g, at least about 250 g oil/100 g, at least about 300 g oil/100 g or at least about 400 g oil/100 g pharmaceutically acceptable material. The oil absorption value is determined as described in the experimental section herein.

The present inventors have found that a common feature of some of the materials suitable for use as oil sorption material is that they have a relatively large surface area. Accordingly, an oil sorption material for use according to the invention may have a BET surface area of at least 5 m²/g such as, e.g., at least about 25 m²/g, at least about 50 m²/g, at least about 100 m²/g, at least about 150 m²/g, at least about 200 m²/g, at least about 250 m²/g or at least about 275 m²/g.

As mentioned above one of the characteristic features of an oil sorption material for use according to the invention is that it retains a good flowability even if it has been loaded with oily material. Thus, the flowability of the pharmaceutically acceptable material loaded with 25% w/w or more such as, e.g. 30% w/w or more, 40% w/w or more, 45% w/w or more, 50% w/w or more, 55% w/w or more, 60% w/w or more, 65% w/w or more or about 70% w/w viscoleo will normally meet the Ph. Eur. requirements.

The inventors have found that suitable oil sorption material can be selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

In a specific embodiment, the oil sorption material comprises silica acid or a derivative or a salt thereof such as, e.g., silicon dioxide or a polymer thereof.

In a further specific embodiment, the oil sorption material is a silicon dioxide product that has properties corresponding to Zeofree® 5161A, Zeofree® 5162, Zeofree® 5175A, Zeopharm® 80 (available from J. M. Huber, Hamina, Finland), Aeroperl® 300, Sident® 22S, Sipernat®160, Sipernat® 160PQ, Sipernat® 22, Sipernat® 22 LS, Sipernat®22, Sipernat® 22 LS, Sipernat® 22S, Sipemat® 2200, Sipernat® 310, Sipernat® 320, Sipernat® 320 DS, Sipernat® 325 C, Sipernat® 35, Sipernat® 350, Sipernat® 360, Sipernat® 383 D8, Sipernat® 44, Sipernat® 44MS, Sipernat® 50, Sipernat® 50S, Sipernat® 50 S, Sipernat® 500 LS, or Sipernat® 570 (available from Degussa, Frankfurt, Germany).

As it appears from the examples herein, a very suitable material is Aeroperl® 300 (including materials with properties like or corresponding to those of Aeroperl® 300).

An oil sorption material according to the invention is very advantageous for use in the preparation of pharmaceutical, cosmetic, nutritional and/or food compositions, wherein the composition comprises oily material. One of the advantages is that is it possible to incorporate a relatively large amount of oil and oily-like material and still have a material that is solid. Thus, it is possible to prepare solid compositions with a relatively high load of oily materials by use of an oil sorption material according to the invention. As mentioned herein before, within the pharmaceutical field it is an advantage to be able to incorporate a relatively large amount of an oily material in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance. Thus, in a specific embodiment it is used in the preparation of pharmaceutical compositions.

The oil sorption material for use in the further processing into solid composition normally absorbs about 5% w/w or more, such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more of an oil or an oily material and is still a solid material.

The concentration of the oil sorption material in the particulate composition is about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more.

Normally, the concentration of the oil sorption material in a solid pharmaceutical particulate composition or a pharmaceutical composition is in a range from about 20% to about 80% w/w such as, e.g., from about 25% to about 75% w/w.

In those cases where a matrix system is desired e.g. a hydrocolloid (examples given herein) may be added before of after the controlled agglomeration process.

The particulate material obtained by a method of the invention may be filled or further processed into a suitable dosage form. To this end, addition of one or more pharmaceutically acceptable excipients may be employed. Suitable excipients include those mentioned herein before and those mentioned below.

Pharmaceutically Acceptable Excipients

A solid pharmaceutical particulate material or a pharmaceutical composition according to the invention may further comprise a pharmaceutically acceptable excipient.

In the present context the terms “pharmaceutically acceptable excipient” are intended to denote any material, which is inert in the sense that it substantially does not have any therapeutic and/or prophylactic effect per se. Such an excipient may be added with the purpose of making it possible to obtain a pharmaceutical, cosmetic and/or foodstuff composition, which have acceptable technical properties.

Examples of suitable excipients for use a particular material or composition according to the invention include fillers, diluents, disintegrants, binders, lubricants etc. or mixture thereof. As the particulate material or composition according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose (e.g. spray-dried lactose, α-lactose, β-lactose, Tabletose®, various grades of Pharmatose®, Microtose® or Fast-Floc®, microcrystalline cellulose (various grades of Avicel®, Elcema®, Vivacel®, Ming Tai® or Solka-Floc®, hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A 15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the second composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in the particulate material or composition are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a particulate material or in a composition according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilizing agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance.

Examples of Surfactants are given in the following.

Suitable excipients for use in a composition or a solid dosage form according to the invention are surfactants such as, e.g., hydrophobic and/or hydrophilic surfactants as those disclosed in WO 00/50007 in the name of Lipocine, Inc. Examples on suitable surfactants are
i) polyethoxylated fatty acids such as, e.g. fatty acid mono- or diesters of polyethylene glycol or mixtures thereof such as, e.g. mono—or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30,

PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000,
ii) polyethylene glycol glycerol fatty acid esters, i.e. esters like the above-mentioned but in the form of glyceryl esters of the individual fatty acids;
iii) glycerol, propylene glycol, ethylene glycol, PEG or sorbitol esters with e.g. vegetable oils like e.g. hydrogenated castor oil, almond oil, palm kernel oil, castor oil, apricot kernel oil, olive oil, peanut oil, hydrogenated palm kernel oil and the like,
iv) polyglycerized fatty acids like e.g. polyglycerol stearate, polyglycerol oleate, polyglycerol ricinoleate, polyglycerol linoleate,
v) propylene glycol fatty acid esters such as, e.g. propylene glycol monolaurate, propylene glycol ricinoleate and the like,
vi) mono- and diglycerides like e.g. glyceryl monooleate, glyceryl dioleae, glyceryl mono- and/or dioleate, glyceryl caprylate, glyceryl caprate etc.;
vii) sterol and sterol derivatives;
viii) polyethylene glycol sorbitan fatty acid esters (PEG-sorbitan fatty acid esters) such as esters of PEG with the various molecular weights indicated above, and the various Tween® series;
ix) polyethylene glycol alkyl ethers such as, e.g. PEG oleyl ether and PEG lauryl ether;
x) sugar esters like e.g. sucrose monopalmitate and sucrose monolaurate;
xi) polyethylene glycol alkyl phenols like e.g. the Triton® X or N series;
xii) polyoxyethylene-polyoxypropylene block copolymers such as, e.g., the Pluronic® series, the Synperonic® series, Emkalyx®, Lutrol®, Supronic® etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407;
xiii) sorbitan fatty acid esters like the Span® series or Ariacel® series such as, e.g. sorbinan monolaurate, sorbitan monopalmitate, sorbitan monooleate, sorbitan monostearate etc.;
xiv) lower alcohol fatty acid esters like e.g. oleate, isopropyl myristate, isopropyl palmitate etc.;
xv) ionic surfactants including cationic, anionic and zwitterionic surfactants such as, e.g. fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0,1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

Methods

Threshold Test

The test involves determination of flowability according to the method described in Ph.Eur. by measuring the flow rate of the material out of a funnel with a nozzle diameter of 10.0 mm.

Viscoleo (medium chain triglycerides MCT; Miglyol 812 N from Condea) was added to 100 g of the solid pharmaceutically acceptable material to be tested for use as an oil sorption material and mixed manually. The mixture obtained was sieved through sieve 0.3 mm to assure a homogenous mixture. The oil was added successively until a flow of 100 g of the mixture could not flow through the nozzle. If the material to be tested has a high bulk volume (e.g. like that of Aeroperl 300) only 50 g of the mixture is used when testing these blends. The maximal concentration of oil where flow of material could be obtained is called the Threshold Value (given as % w/w).

Release Test

A fat-soluble colorant Sudan II (BDH Gur®) obtained from BDH VWR International 14.3 mg was dissolved in 50.0 g visco!eo (fractionated medium chain triglycerides).

10 g of the oil was added to 10.0 g of the solid pharmaceutically acceptable material to be tested for use as an oil sorption material and mixed until the oil was fully absorbed in the solid material. The mixture was subsequently sieved through sieve 0.3 mm to achieve a homogeneous mixture.

1.00 g of the mixture was transferred to a centrifugal tube and 3.00 ml of water was added. The suspension was mixed in a blood sample turner for 1 hour and subsequently centrifuged for 10 minutes at 5000 rpm. The upper phase of oil and water was transferred carefully to a beaker and the water was evaporated in an oven at 80° C. until constant weight. The amount of oil released from the solid material was calculated on basis of the weight of the remaining after evaporation of the water phase.

Disintegration Test

The disintegration time was determined according to the method described in to Ph. Eur.

Determination of Bulk Density

The bulk density was measured by pouring 100 g of the powder in question in a 250 ml graduated cylinder. The bulk density is given as the tapped bulk density in g/mi. The determination was performed according to Ph. Eur. (apparent volume).

Determination of Oil Absorption Value

The oil absorption value is determined by adding well-defined amounts (a 10 g) of viscoleo to a well-defined amount of the oil sorption material (100 g) to be tested. The oil absorption value (expressed as g viscoleo/100 g material) is reached when a further addition of 10 g oil results in a material that does not have suitable properties with respect to flowability, i.e. the material does not meet the meet the requirements when tested according to Ph.Eur. (flowability test; see above under Threshold Test herein).

Determination of BET Surface Area

The apparatus applied was a Micromertics Gemini 2375. The method applied was according to USP volumetric methods based on multiple point determination.

Determination of Flowability

The flowability was determined according to the method described in Ph. Eur. measuring the flow rate of the material out of a funnel with a nozzle diameter of 10.0 mm.

Determination of Weight Variation

The tablets prepared in the Examples herein were subject to a test for weight variation performed in accordance with Ph. Eur.

Determination of Average Tablet Hardness

The tablets prepared in the Examples herein were subject to at test for tablet hardness employing Schleuniger Model 6D apparatus and performed in accordance with the general instructions for the apparatus.

### EXAMPLES

### Example 1

Modified Release Polydepot Capsule Based on Intragranular Swelling Hydrocolloid Matrix of Hydroxypropylcellulose

This Example illustrates a process as outlined in FIG. 1. This Example illustrates incorporation of a swellable hydrocolloid in the granules by means of the controlled agglomeration process. Optionally, after addition of one or more pharmaceutically acceptable excipients, the obtained powder may be filled into e.g. gelatine capsules or may be further processed into tablets.

| Substance | % | mg |
|---|---|---|
| Tacrolimus | 0.50 | 1.00 |
| HPMC | 20.00 | 40.00 |
| Lactose 200 mesh | 30.00 | 60.00 |
| PEG 6000 | 34.65 | 69.30 |
| Poloxamer 188 | 14.85 | 29.70 |
| Total | 100.00 | 200.00 |

Tacrolimus was dissolved in Polyethylene glycol 6000 and Poloxamer 188 (70:30 w/w ratio) at 70° C. The solution was sprayed on a mixture of 150 lactose and 100 g HPMC in a fluid bed Strea-1 maintaining the temperature of the solution at about 70° C. The granular product obtained was sieved through sieve 0.7 mm and filled into hard gelatine capsules (200 mg).

### Example 2

Modified Release Matrix Tablet Based on Swelling Hydrocolloid Matrix of Hydroxypropylcellulose in the Extra-granular Phase

This Example illustrates the process outlined in FIG. 2: A swellable hydrocolloid matrix is formed by adding a hydrocolloid to the granules obtained by the controlled agglomeration process. Optionally, after addition of one or more pharmaceutically acceptable excipients, the obtained powder may be filled into e.g. gelatine capsules or may be further processed into tablets.

| Substance | % | mg |
|---|---|---|
| Tacrolimus | 0.50 | 1.00 |
| HPMC | 19.90 | 40.00 |
| Lactose 200 mesh | 29.85 | 60.00 |
| PEG 6000 | 34.48 | 69.30 |
| Poloxamer 188 | 14.78 | 29.70 |
| Magnesium stearate | 0.50 | 1.01 |
| Total | 100.00 | 201.01 |

Tacrolimus was dissolved in Polyethylene glycol 6000 and Poloxamer 188 (70:30 w/w ratio) at 70° C. The solution was sprayed on 250 g lactose in a fluid bed Strea-1 maintaining the temperature of the solution at about 70° C. The granular product obtained was sieved through sieve 0.7 mm and blended with HPMC and magnesium stearate for 0.5 min in a Turbula mixer.

The mixture was compressed into 8 mm tablets of 1 mg strength (200 mg tablet with compound cup shaped.

Mean disintegration time: 20 min, Hardness: 45 N

### Example 3

Modified Release Matrix Tablet Based on Lipophilic Matrix of Glyceryl Mono-Stearate

This Example illustrates the process outlined in FIG. 3. In this process the drug substance, illustrated with tacrolimus as an example, is dissolved in the composition that contains an oily material and which while heated is sprayed on a second composition contained in e.g. a fluid bed.

Optionally after addition of one or more pharmaceutically acceptable excipients, the granules obtained may be filled into e.g. gelatine capsules or compressed into tablets.

| Substance | % | mg |
|---|---|---|
| Tacrolimus | 0.50 | 1.00 |
| Lactose 200 mesh | 49.75 | 100.00 |
| Glyceryl monostearate | 49.25 | 99.00 |
| Magnesium stearate | 0.50 | 1.01 |
| | 100.00 | 201.01 |

Tacrolimus was dissolved in glyceryl monostearate at 70° C. The solution was sprayed on 250 g lactose in a fluid bed Strea-1 maintaining the temperature of the solution at about 70° C. The granular product obtained was sieved through sieve 0.7 mm and blended with magnesium stearate for 0.5 min in a Turbula mixer.

The mixture was compressed into 8 mm tablets with strength of 1 mg (200 mg tablet with compound cup shaped.

Mean disintegration time: 20 min, Hardness: 45 N

### Example 4

Multiparticulate Modified Release Formulation Based on Coating

| Substance | % |
|---|---|
| Danazol | 2.00 |
| PEG 6000 | 34.65 |
| Poloxamer | 14.85 |
| Lactose 200 Mesh | 48.50 |
| Total | 100.00 |

4.0% w/w danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and subsequently coated with a semipermeable membrane and filled into hard gelatine capsules.

Different types of coatings were applied. The details follow as Examples 4a, 4b and 4c.

### Examples 4a

Surelease® Coating of CA (Controlled Agglomeration) Generated Particles

250 g of granules prepared as described above is coated with a Surelease® coating by applying 1 kg of the following coating mixture per 250 g granules. The coating mixture is prepared by diluting Surelease® to 12.5% w/w with water. The coating mixture is applied on the granules by means of the same apparatus used for making the granules, the Strea 1 equipped with a Wurster insert using the following conditions:
Nozzle position: bottom
Inlet air temperature: 75-80° C.
Product temperature: approx. 28° C.
Nozzle pressure: 2.5-3.0 bar
Spraying rate: 12 g/min
Fluidized air velocity: 20-25 m³/hour

In order to obtain a film thickness of about 10 μm, an amount of polymer corresponding to about 57% of the weight of the granules should be employed.

In the same manner as described above, coated granules were prepared by use of various amounts of coating mixture in order to obtain granules having various amounts of film coating applied (i.e. 2%, 10%, 20%, 30%, 40%, and 50% w/w, respectively). In order to obtain a coating of 50% w/w, 1 kg of Surelease® diluted to 12.5% wfw was employed per 250 g. granules. The thus coated granules were subjected to a dissolution test in order to test the release rate of Danazole versus the thickness of the film.

The coated Danazole granules were subjected to a dissolution test employing in each of the six vessels a dose corresponding to 100 mg of danazole of the granules and 900 ml of phosphate buffer solution pH 7.5, USP as dissolution medium. A Sotax USP apparatus was employed. The dissolution test was performed in accordance with USP, method 2 (paddle-method) and 50 rpm using a phosphate buffer solution, pH 7.5 (USP) as dissolution medium and a temperature of 37° C. In some cases the dissolution medium was 0.1 N hydrochloric acid during the first 2 hours of testing; then the medium was adjusted to pH 6.8 by addition of Na₃ PO₄.

900 ml of dissolution medium was placed into each of the 6 vessels of the Sotax apparatus employed. The temperature was controlled thermostatically at 37° C.±0.5° C. In those cases where the sample under testing was a tablet, one tablet was placed into each vessel and the test was started. In those cases where the sample under testing was a sample of a particulate formulation according to the invention, an accurately weighted amount corresponding to one dose of the active substance was placed in each vessel and the test was started. At appropriate intervals a 10 ml sample was removed from each vessel for individual measurement (and replaced with another 10 ml of dissolution medium). The samples were filtered and cooled to room temperature and analyzed.

The following results were obtained (the values given are the mean values of two determinations and the values are given as the weight percentages released after the stated time period):

TABLE 1

| | % film coating | | | | | |
|---|---|---|---|---|---|---|
| time (hours) | 2% | 10% | 20% | 30% | 40% | 50% |
| 0.5 | 94.2 | 81.9 | 36.3 | 12.6 | 8.5 | 9.6 |
| 1.0 | 96.3 | 94 | 57.1 | 20.3 | 13.7 | 13.7 |
| 2.0 | 97.2 | 97.2 | 83.7 | 36.7 | 23.5 | 21.9 |
| 3.0 | 101 | 101 | 97.2 | 51.9 | 35.5 | 31.8 |
| 4.0 | 99.3 | 101 | 101 | 63.7 | 45 | 41.6 |
| 5.0 | | 99.7 | 102 | 76.3 | 56 | 48.6 |
| 6.0 | | 99.8 | 102 | 86.2 | 63.8 | 57.5 |
| 24 | | 99.9 | 104 | 106 | 101 | 98.4 |

The results clearly show that the granules prepared by controlled agglomeration are sufficiently robust to withstand a coating procedure and that a modified release coating can be made. Furthermore, the results show that the retardation in release increases as the coating thickness increases. Granules coated with 2% w/w or 10% w/w Surelease® release almost instantly the total amount of danazole contained in the granules

### Example 4b

### Ethyl Cellulose Coating of Granules Prepared by Controlled Agglomeration Technique

250 g of granules prepared as described above are coated with an ethyl cellulose coating by applying 625 g of the following coating mixture per 250 g granules. The coating mixture is prepared by dissolving 10% of ethylcellulose 20 cps in ethanol and adding 8% w/w DBS (dibutylsebacate) as a plasticizer (625 g coating solution per 250 g granules have the following composition:

| | |
|---|---|
| Ethanol | 560 g |
| Ethocel ® | 60 g |
| Dibuthylsebacate | 5 g |

The content corresponds to 9.9% w/w Ethoce® as dry matter and 0.8% w/w dibuthylsebacetate as dry matter).

The coating solution is applied on the granules by means of the controlled agglomeration apparatus (Strea 1′ equipped with a Wurster insert) using the following conditions:
Nozzle position: bottom
Inlet air temperature: 50-65° C.
Product temperature: 28-35° C.
Nozzle pressure: 3.0 bar
Spraying rate: 15 g/min.
Fluidized air velocity: 20-22 m³/hour

A film coating having a thickness of about 5 μm is obtained. 625 kg coating solution per 250 g granules is applied, corresponding to 112.5 g dry matter per 250 g granules (45% w/w). In the same manner as described above, coated granules were prepared by use of various amounts of coating mixture in order to obtain granules having various amounts of film coating applied (i.e. 8.6%, 11.9%, 16.2%, 20.5%, 24.8%, and 27% wh, respectively). The thus coated granules were subjected to a dissolution test in order to test the release rate of Danazole versus the thickness of the film.

The coated Danazole granules were subjected to a dissolution test employing in each of the vessels 100 mg of danazole of the granules and 900 ml of phosphate buffer solution pH 7.5, USP as dissolution medium (see above for details).

The following results were obtained (the values given are the mean values of two determinations and the values given are the weight percentages released after the stated time period):

| | % film coating | | | | | |
|---|---|---|---|---|---|---|
| time (hours) | 8.6% | 11.9% | 16.2% | 20.5% | 24.8% | 27% |
| 0.5 | 34.3 | 34.5 | 7.1 | 3.1 | 2.6 | 1.1 |
| 1.0 | 48.7 | 42.7 | 12.4 | 4.8 | 3.5 | 1.9 |
| 2.0 | 63.7 | 55.4 | 20.1 | 8.8 | 6.8 | 4.3 |
| 3.0 | 73 | 69.2 | 27.2 | 11.7 | 9.3 | 6.4 |
| 4.0 | 76.2 | 70.9 | 29.6 | 12.7 | 10.1 | 7 |
| 5.0 | 79.7 | 73.1 | 33.5 | 14.2 | 11.8 | 8.7 |
| 6.0 | 80.3 | 74.5 | 36.8 | 16.4 | 13.7 | 10.6 |
| 24 | 98.7 | 83.5 | 62.1 | 36.2 | 26 | 21 |

The results clearly show that the granules prepared by controlled agglomeration are sufficiently robust to withstand a coating procedure and that a modified release coating can be obtained. Furthermore, the results show that the retardation in release increases as the coating thickness increases. Granules coated with 8.6% w/w or 11.9% w/w ethylcellulose display also a modified release pattern. The results also show that less film is needed when using an ethanol-based film than when an aqueous based film is used. This is most likely due to the dissolution characteristics of ethylcellulose in ethanol as ethylcellulose easily dissolves in ethanol and thus perform a more homogeneous and tight film on the granules. In the case of an aqueous based film, the polymer (i.e. ethyl-cellulose) is dispersed in the medium as small particles (dispersion), which makes the coating more difficult.

### Example 4c

Eudragit® Coating of Granules

0.25 kg of granules prepared as described above is coated with 0.610 kg of the following coating mixture containing Eudragit® RS 30D, 30% w/w dispersion in water:

| | |
|---|---|
| Eudragit ® RS 30D (30% w/w dispersion) (corresponding | 475.0 g |
| to 142.5 g dry matter) | |
| Triethyl citrate (Eudraflex ®) | 28.5 g |
| Microtalcum | 71.3 g |
| Antifoam M 10 | 3.0 g |
| Purified water | 640.0 g |

The coating mixture is applied on the granules by means of a controlled agglomeration apparatus (Strea 1 equipped with a Wurster insert) using the following conditions:
Nozzle position: bottom
Nozzle size 0.8 mm
Inlet air temperature: 60° C.-75° C.
Product temperature: 25° C.-34° C.
Nozzle pressure: 2 bar
Spraying rate: up to 9 g/min
Fluidized air velocity: up to 25 m³/hour

A film coating having a thickness of about 10 μm is obtained. About 43% w/w dry matter is applied on the granules. The thus coated granules were subjected to a dissolution test in order to test the release rate of danazole versus time.

The coated danazole granules were subjected to a dissolution test employing in each of the vessels a dose corresponding to 300 mg of danazole of the granules and 900 ml of 0.1 N hydrochloric acid as dissolution medium. After 2 hours the pH of the dissolution medium was adjusted to pH 6.8 by addition of Na₃PO₄ (see above).

The following results were obtained (the values given are the mean values of two determinations and the values given are weight percentages released after the stated time period):

| | % film coating |
|---|---|
| time (hours) | 43% |
| 0.5 | 6.1 |
| 1.0 | 9.8 |
| 2.0 | 17.8 |
| 3.0 | 21.0 |
| 4.0 | 27.1 |
| 5.0 | 31.3 |
| 6.0 | 35.3 |
| 24 | 80.8 |

Alternatively granule products might be compressed into a tablet followed by coating of the tablet with a film membrane or compression coating of the tablet core.

### Example 5

Tablet Modified Release Formulation Based on Coating

| Substance | % | mg |
|---|---|---|
| Danazol | 2.00 | 5.00 |
| PEG 6000 | 34.65 | 86.63 |
| Poloxamer 188 | 14.85 | 37.13 |
| Lactose 200 Mesh | 47.50 | 118.75 |
| Magnesium stearate | 1.00 | 2.50 |
| Total | 100.00 | 250.00 |

4.0% of Danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and mixed with 1% magnesium stearate for 0.5 min in a Turbula mixer. 8 mm tablets (compound cup) were compressed on a Korsch EKO with a weight of 250 mg and a strength of 5 mg. Mean tablet hardness: 75 N. The tablets were subsequently coated with a semipermeable membrane as in Example 4a (Surelease coating).

### Example 6

Delayed Release Tablet Formulation

Tablet composition:

| Substance | % | mg |
|---|---|---|
| Danazol | 2.00 | 5.00 |
| PEG 6000 | 34.65 | 86.63 |
| Poloxamer 188 | 14.85 | 37.13 |
| Lactose 200 Mesh | 47.50 | 118.75 |
| Magnesium stearate | 1.00 | 2.50 |
| Total | 100.00 | 250.00 |

4.0% of Danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and mixed with 1% magnesium stearate for 0.5 min in a Turbula mixer. 8 mm tablets (compound cup) were compressed on a Korsch EKO with a weight of 250 mg and a strength of 5 mg. Mean tablet hardness: 75 N. The tablets were subsequently enteric coated with an aqueous based latex suspension of Eudragit L30D (methacrylic acid co-polymer), Rbhm Pharma. The film composition is shown below

Film composition:

| Substance | % |
|---|---|
| Eudragit L30D | 40.0 |
| Water | 52.0 |
| Triethyl citrate | 1.8 |
| Silicon oil | 0.2 |
| Talc | 6.0 |

400 g tablets were coated in a Strea-1 with a Wurster insert (bottom spray) using the following process conditions: Liquid flow rate: 7 g/min, inlet air temperature 60-65° C. Product temperature: 29-31° C. Outlet air temp: 26-28° C. Inlet air flow: 18-25 m³/hour. The tablets were coated until a weight gain of 4% was obtained and cured for 24 hours at 30° C.

### Example 7

Tablet composition:

| Substance | % | mg |
|---|---|---|
| Danazol | 1.91 | 10.05 |
| Metolose HS 90 100 cp | 20.86 | 109.53 |
| Lactose 200 mesh | 31.30 | 164.30 |
| PEG 6000 | 32.15 | 168.78 |
| Poloxamer 188 | 13.78 | 72.33 |
| Total | 100.00 | 525.00 |

Matrix Tablet with Intragranular Hydrocolloid Tablet composition:

4.0% of Danazol was dissolved in a melted mixture of polyethyleneglycol 6000 and Poloxamer 188 (70:30) at 90° C. 318 g of the solid dispersion was sprayed on a mixture of 150 g of lactose and 100 g Metolose 90SH 100 cP in a fluid bed Strea-1. The granular product was sieved through sieve 0.7 mm. The product was directly compressed into 12 mm tablets (compound cup) on a Diaf TM20. The tablets had a mean weight of 525 mg and a strength of 10 mg. Mean tablet hardness: 52 N.

### Example 8

Multi-Particulate Modified Release Capsule with Intragranular Hydrocolloid

Capsule composition:

| Substance | % | mg |
|---|---|---|
| Danazol | 1.91 | 10.05 |
| Metolose HS 90 100000 cp | 20.86 | 109.53 |
| Lactose 200 mesh | 31.30 | 164.30 |
| PEG 6000 | 32.15 | 168.78 |
| Poloxamer 188 | 13.78 | 72.33 |
| Total | 100.00 | 525.00 |

4.0% of Danazol was dissolved in a melted mixture of polyethyleneglycol 6000 and Poloxamer 188 (70:30) at 90° C. 318 g of the solid dispersion was sprayed on a mixture of 150 g of lactose and 100 g Metolose 90SH 100000 cP in a fluid bed Strea-1. The granular product was sieved through sieve 0.7 mm. and filled into hard gelatine capsules (525 mg)

### Example 9

Matrix Tablet with Extragranular Hydrocolloid

Tablet composition:

| Substance | % | mg |
|---|---|---|
| Danazol | 1.61 | 10.00 |
| Lactose 200 mesh | 38.14 | 237.5 |
| PEG 6000 | 27.83 | 173.3 |
| Poloxamer 188 | 11.93 | 74.3 |
| Metolose HS 90 15000 cP | 20.00 | 124.5 |
| Magnesium stearate | 0.5 | 3.1 |
| Total | 100.00 | 623 |

The granular product from Example 4 was mixed with 20% Metolose 90 SH 15000 cP in a turbula mixer for 3 minutes and subsequently mixed with 0.5% magnesium stearate for 0.5 min. The granulate was directly compressed into 12 mm tablets (compound cup) on a Diaf TM20. The tablets had a mean weight of 623 mg and a strength of 10 mg.

Mean tablet hardness: 41 N.

1. A method for the preparation of a pharmaceutical particulate composition for modified release of one or more therapeutically, prophylactically and/or diagnostically active substances, the method comprising
spraying a first composition comprising an oily material, which has a melting point of about 5° C. or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C., at least about 5° C. or at least about 10° C. lower than the melting point of the oily material and/or of the first composition,
optionally, mixing or other means of mechanical working the second composition onto which the first composition is sprayed to obtain a particulate material,
optionally, adding one or more release-rate modifier,
mixing or other means of mechanical working the second composition—including, if relevant, the added one or more release-rate modifying substances—onto which the first composition is sprayed to obtain a particulate composition,
the particulate composition comprising a sufficient amount of at least one release-rate modifier to provide a modified release of the active substance sufficient to provide a duration of therapeutic, prophylactic and/or diagnostic effect of at least about 2 hours such as, e.g., at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 15 hours, at least about 17 hours, at least about 20 hours, at least about 22 hours or at least about 24 hours when the composition is exposed to an aqueous environment.

2. A method for the preparation of a pharmaceutical particulate composition for modified release of one or more therapeutically, prophylactically and/or diagnostically active substances, the method comprising
spraying a first composition comprising an oily material, which has a melting point of about 5° C. or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C., at least about 5° C. or at least about 10° C. lower than the melting point of the oily material and/or of the first composition,
optionally, mixing or other means of mechanical working the second composition onto which the first composition is sprayed to obtain a particulate material,
optionally, adding one or more release-rate modifier,
mixing or other means of mechanical working the second composition—including, if relevant, the added one or more release-rate modifying substances—onto which the first composition is sprayed to obtain a particulate composition,
the particulate composition comprising a sufficient amount of at least one release-rate modifier to provide a modified release of the active substance sufficient to provide a dissolution rate in vitro of the particulate composition, which—when measured according to USP dissolution test (paddle) employing water as dissolution medium, 100 rpm and a temperature of about 37° C. permits release of less than 85% w/w within about 30 min after start of the test.

3. A method according to claim 2, wherein less than about 80% w/w such as, e.g., less than about 75% w/w, less than about 70% w/w, less than about 65% w/w, less than about 60% w/w, less than about 55% w/w, less than about 50% w/w, less than about 45% w/w, less than about 40% w/w, less than about 35% w/w, less than about 30% w/w or less than about 25% w/w is released within about 30 min after start of the test.

4. A method according to claim 2, wherein less than 85% w/w is released within the first hours, within about 2 hours, within about 3 hours, within about 4 hours, within about 5 hours or within about 6 hours after start of the test.

5. A method according to claim 4, wherein less than 80% w/w is released such as, e.g., less than about 75% w/w, less than about 70% w/w, less than about 65% w/w, less than about 60% w/w, less than about 55% w/w, less than about 50% w/w or less than about 45% w/w is released within the first hour after start of the test.

6. A method according to claim 4, wherein less than 80% w/w is released such as, e.g., less than about 75% w/w, less than about 70% w/w, less than about 65% w/w, less than about 60% w/w, less than about 55% w/w or less than about 50% w/w is released within 2 hours after start of the test.

7. A method according to claim 4, wherein less than 80% w/w is released such as, e.g., less than about 75% w/w, less than about 70% w/w, less than about 65% w/w, less than about 60% w/w, less than about 55% w/w or less than about 50% w/w is released within 3 hours after start of the test.

8. A method according to claim 4, wherein less than 80% w/w is released such as, e.g., less than about 75% w/w, less than about 70% w/w, less than about 65% w/w or less than about 60% w/w is released within 6 hours after start of the test.

9. A method according to claim 2, wherein less than 75% w/w is released within about 7 hours, within about 8 hours, within about 9 hours, within about 10 hours, within about 11 hours or within about 12 hours after start of the test.

10. A method according to claim 9, wherein less than 70% w/w or less than about 65% w/w is released within about 7 hours, within about 8 hours, within about 9 hours, within about 10 hours, within about 11 hours or within about 12 hours after start of the test.

11. A method according to claim 9, wherein more than 20% w/w such as, e.g., more than about 25% w/w, more than about 30% w/w, more than about 35% w/w or more than about 40% w/w is released within about 7 hours, within about 8 hours, within about 9 hours, within about 10 hours, within about 11 hours or within about 12 hours after start of the test.

12. A method according to any of claim 2, wherein more than 20% w/w such as, e.g., more than about 25% w/w, more than about 30% w/w, more than about 35% w/w, more than about 40% w/w, more than about 45% w/w, more than about 50% w/w, more than about 55% w/w or more than about 60% w/w is released within about 15 hours, within about 20 hours or within about 24 hours after start of the test.

13. A method for the preparation of a pharmaceutical particulate composition for modified release of one or more therapeutically, prophylactically and/or diagnostically active substances, the method comprising
spraying a first composition comprising an oily material, which has a melting point of about 5° C. or more such as, e.g., about 10° C. or more, about 20° C. or more or about 25° C. or more and which is present in the first composition in liquid form, on a second composition comprising a material in solid form, the second composition having a temperature of at the most a temperature corresponding to the melting point of the oily material and/or of the first composition such as, e.g., a temperature of at least about 2° C., at least about 5° C. or at least about 10° C. lower than the melting point of the oily material and/or of the first composition,
optionally, mixing or other means of mechanical working the second composition onto which the first composition is sprayed to obtain a particulate material,
optionally, adding one or more release-rate modifier,
mixing or other means of mechanical working the second composition—including, if relevant, the added one or more release-rate modifying substances—onto which the first composition is sprayed to obtain a particulate composition,
the particulate composition comprising a sufficient amount of at least one release-rate modifier so that following ingestion by a subject in need thereof the active substance is released in the gastrointestinal tract of the mammal at a rate so that less than 85% w/w is released within the first 30 min after ingestion.

14. A method according to claim 13, wherein less than about 80% w/w such as, e.g., less than about 75% w/w, less than about 70% w/w, less than about 65% w/w, less than about 60% w/w, less than about 55% w/w, less than about 50% w/w, less than about 45% w/w, less than about 40% w/w, less than about 35% w/w, less than about 30% w/w or less than about 25% w/w is released within about 30 min after ingestion.

15. A method according to claim 13, wherein less than 85% w/w is released within the first hours, within about 2 hours, within about 3 hours, within about 4 hours, within about 5 hours or within about 6 hours after ingestion.

16. A method according to claim 15, wherein less than 80% w/w is released such as, e.g., less than about 75% w/w, less than about 70% w/w, less than about 65% w/w, less than about 60% w/w, less than about 55% w/w, less than about 50% w/w or less than about 45% w/w is released within the first hour after ingestion.

17. A method according to claim 15, wherein less than 80% w/w is released such as, e.g., less than about 75% w/w, less than about 70% w/w, less than about 65% w/w, less than about 60% w/w, less than about 55% w/w or less than about 50% w/w is released within 2 hours after ingestion.

18. A method according to claim 15, wherein less than 80% w/w is released such as, e.g., less than about 75% w/w, less than about 70% w/w, less than about 65% w/w, less than about 60% w/w, less than about 55% w/w or less than about 50% w/w is released within 3 hours after ingestion.

19. A method according to claim 15, wherein less than 80% w/w is released such as, e.g., less than about 75% w/w, less than about 70% w/w, less than about 65% w/w or less than about 60% w/w is released within 6 hours after ingestion.

20. A method according to claim 13, wherein less than 75% w/w is released within about 7 hours, within about 8 hours, within about 9 hours, within about 10 hours, within about 11 hours or within about 12 hours after ingestion.

21. A method according to claim 20, wherein less than 70% w/w or less than about 65% w/w is released within about 7 hours, within about 8 hours, within about 9 hours, within about 10 hours, within about 11 hours or within about 12 hours after ingestion.

22. A method according to claim 13, wherein more than 20% w/w such as, e.g., more than about 25% w/w, more than about 30% w/w, more than about 35% w/w or more than about 40% w/w is released within about 7 hours, within about 8 hours, within about 9 hours, within about 10 hours, within about 11 hours or within about 12 hours after ingestion.

23. A method according to aly of claim 13, wherein more than 20% w/w such as, e.g., more than about 25% w/w, more than about 30% w/w, more than about 35% w/w, more than about 40% w/w, more than about 45% w/w, more than about 50% w/w, more than about 55% w/w or more than about 60% w/w is released within about 15 hours, within about 20 hours or within about 24 hours after ingestion.

24. A method according to claim 1,
wherein the bioavailability (measured as AUCO_{0-∝}) of the active substance after oral administration of the particulate composition to a subject is at least about 50% such as, e.g., at least about 55%, at least about 60%, at least about 65%, at least about 70%, at least about 75%, at least about 80%, at least about 80%, at least about 85%, at least about 90% compared to the bioavailability of the active substance after oral administration of a particulate composition obtained in analogues matter but without any release-rate modifier.

25. A method according to ally of the preceding claim 1,
wherein the active substance has a bioavailability of less than about 50% when administered to a subject in the form of plain tablets.

26. A method according to any of the preceding claim 2,
wherein the active substance has a water-solubility at room temperature of at the most about 10 mg/ml such as, e.g., at the most about 7.5 mg/ml, at the most about 6 mg/ml, at the most about 5 mg or at the most about 4 mg/ml.

27. A method according to aly of the preceding claim 1,
wherein the active substance has a water-solubility at room temperature of at the most about 3 mg/ml such as, e.g., at the most about 2 mg/ml, at the most about 1 mg/ml, at the most about 750 μg/ml, at the most about 500 μg/ml, at the most about 250 μg/ml, at the most about 100 μg/ml, or at the most about 50 μg/ml, or at the most about 25 μg/ml, or at the most about 20 μg/ml or or at the most about 10 μg/ml.

28. A method according to any of the preceding claim 1,
wherein the active substance has a t_{1/2} in plasma of at the most about 8 hours.

29. A method according to claim 1,
wherein the active substance is subject to first-pass metabolism.

30. A method according to claims 1,
wherein the active substance is subject to degradation in the gastrointestinal tract.

31. A method according to claim 1,
wherein the active substance is subject to enzymatic degradation is the stomach, duodenum and/or proximal part of ileum.

32. A method according to claim 1,
wherein the active substance is subject to food effect.

33. A method according to claim 1,
wherein a release-rate modifier is present in the first composition.

34. A method according to claim 1,
wherein a release-rate modifier is present in the second composition.

35. A method according to claim 1,
wherein step iii) is included and the release-rate modifier is added to the second composition after the first composition has been applied thereto.

36. A method according to claim 1,
wherein the particulate material obtained has a geometric weight mean diameter d_{gw} of ≧10 μm such as, e.g. ≧20 μm, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

37. A method according to claim 1,
wherein step iii) is included and the release-rate modifier is sprayed to the second composition after the first composition has been applied thereto.

38. A method according to claim 37, wherein the release-rate modifier is applied in the form of a coating composition.

39. A method according to claim 1,
wherein the method is carried out in a high or low shear mixer or in a fluid bed.

40. A method according to claim 1,
wherein the process is carried out in a fluid bed and the spraying of the first composition is performed on the second composition in a fluidised state.

41. A method according to claim 40, wherein the spraying is performed through a spraying device equipped with temperature controlling means.

42. A method according to claim 1, the method being a one-pot method.

43. A method according to claim 1,
wherein the concentration of the oily material in the particulate material is from about 5 to about 95% v/v such as, e.g. from about 5 to 90% v/v, from about 5 to about 85% v/v, from about 5 to about 80% v/v, from about 10 to about 75% v/v, from about 15 to about 75% v/v, from about 20 to abut 75% v/v, from about 25% to about 75% v/v, from about 30% to about 75% v/v, from about 35% to about 75% v/v, from about 25% to about 70% v/v, from about 30% to about 70% v/v, from about 35% to abut 70% v/v, from about 40% to about 70% v/v, from about 45% to about 65% v/v or from about 45% to about 60% v/v.

44. A method according to claim 1,
wherein the oil or oily like material is brought on liquid form by heating the first composition to a temperature, which causes the oily material to melt.

45. A method according to claim 44, wherein the first composition in liquid form has a viscosity (Brookfield DV-III) of at the most about 800 mPas at a temperature of at the most 100° C. such as, e.g., at the most 700, at the most 600, at the most 500 mPas.

46. A method according to claim 2,
wherein the first composition is essentially non-aqueous and it contains at the most about 20% w/w water such as at the most about 15% w/w, at the most abut 10% w/w, at the most about 5% w/w or at the most about 2.5% w/w.

47. A method according to claim 1,
wherein the oily material has a melting point of at least about 30° C. such as, e.g., at least about 35° C. or at least about 40° C.

48. A method according to claim 1,
wherein the oily material has a melting point of at the most about 300° C. such as, e.g., at the most about 250° C., at the most about 200° C., at the most about 150° C. or at the most about 100° C.

49. A method according to claim 1,
wherein the first composition comprises one or more pharmaceutically acceptable excipients.

50. A method according to claim 1,
wherein the second composition comprises one or more pharmaceutically acceptable excipients.

51. A method according to claim 49, wherein the pharmaceutically acceptable excipient is selected from the group consisting of fillers, binders, disintegrants, glidants, colouring agents, taste-masking agents, pH-adjusting agents, solubilizing agents, stabilising agents, wetting agents, surface active agents, antioxidants etc.

52. A method according to claim 1,
wherein the first and/or second composition comprises one or more, the same or different, therapeutically, prophylactically and/or diagnostically active substances.

53. A method according to claim 1,
wherein an active substance is dispersed such as, e.g., dissolved in the first composition.

54. A method according to claim 1 further comprising a step of processing the particulate composition obtained optionally together with one or more pharmaceutically acceptable excipients into a solid dosage form.

55. A method according to claim 54, wherein the solid dosage form is selected from tablets, capsules, sachets and the like.

56. A method according to claim 54, wherein the solid dosage form is provided with a coating.

57. A method according to claim 56, wherein the coating is selected from film-coatings, modified release coatings, enteric coatings, sugar coatings, taste-masking coatings etc.

58 (canceled)

59. A method for preparing a solid composition comprising a drug substance and a release-rate modifying substance, the method comprising the steps of
i) selecting a first composition comprising an oily material having a melting point of at least 5° C.,
ii) optionally bringing the first composition in liquid form,
iii) dispersing or dissolving a drug substance in the liquid first composition at a temperature below the melting point of the drug substance,
iv) spraying the resulting first composition onto a solid second composition having a temperature below the melting point of the first composition,
v) adding a release-modifying substance to the resulting composition
vi) mechanically working the composition to obtain particles, i.e. a particulate material, and
vii) optionally subjecting the particulate material to conventional methods for preparing solid dosage forms.

60-63. (canceled)

64. A pharmaceutical particulate composition for modified release of one or more therapeutically prophylactically and/or diagnostically active substances obtainable by a process as claimed in claim 1.

65. A pharmaceutical composition according to claim 64 in the form of a fluid, semi-solid or solid composition.

66. A pharmaceutical composition according to claim 65 in the form of powders, tablets, capsules or sachets.

67. A pharmaceutical composition according to claim 65 in the form of a liquid such as, e.g., a solution or a dispersion including an emulsion and a suspension.

68 (canceled)

69. A method according to claim 50, wherein the pharmaceutically acceptable excipient is selected from the group consisting of fillers, binders, disintegrants, glidants, colouring agents, taste-masking agents, pH-adjusting agents, solubilizing agents, stabilising agents, wetting agents, surface active agents, antioxidants etc.

70. A pharmaceutical particulate composition for modified release of one or more therapeutically, prophylactically and/or diagnostically active substances obtainable by a process as claimed in claim 2.
